SEQUENCE LISTING (57 sequences)

SEQ ID NO: 1 - DNA, 23 bp, Artificial Sequence
  F PRIMER FOR ARTS (Sept4_i2)
gagacgagagtggcctgaaccga

SEQ ID NO: 2 - DNA, 23 bp, Artificial Sequence
  R PRIMER FOR ARTS (Sept4_i2)
aacaggaacctgtgaccacctgc

SEQ ID NO: 3 - DNA, 24 bp, Artificial Sequence
  F PRIMER FOR beta-Actin
catcctgcgtctggacctggctgg

SEQ ID NO: 4 - DNA, 24 bp, Artificial Sequence
  R PRIMER FOR beta-Actin
ctagaagcatttgcggtggacgat

SEQ ID NO: 5 - DNA, 23 bp, Artificial Sequence
  F PRIMER FOR H5 (Sept4_i1)
gaggacaggactgaagctgggat

SEQ ID NO: 6 - DNA, 23 bp, Artificial Sequence
  R PRIMER FOR H5 (Sept4_i1)
ggttgcaaagcccacatactcct

SEQ ID NO: 7 - DNA, 23 bp, Artificial Sequence
  F PRIMER FOR AFP
tgaaaaccctcttgaatgccaag

SEQ ID NO: 8 - DNA, 23 bp, Artificial Sequence
  R PRIMER FOR AFP
gccacaggccaatagtttgtcct

SEQ ID NO: 9 - DNA, 20 bp, Artificial Sequence
  F PRIMER FOR Omi/Htr2A
acatccggcattgttagctc

SEQ ID NO: 10 - DNA, 20 bp, Artificial Sequence
  R PRIMER FOR Omi/Htr2A
ttcttcttttccccacgatg

SEQ ID NO: 11 - DNA, 20 bp, Artificial Sequence
  F PRIMER FOR Smac/Diablo
ggaaagcagaaaccaagctg

SEQ ID NO: 12 - DNA, 20 bp, Artificial Sequence
  R PRIMER FOR Smac/Diablo
aaaatgctttgggtgtgagg

SEQ ID NO: 13 - DNA, 20 bp, Artificial Sequence
  F PRIMER FOR Siah1
tgactatgtgttaccgccca

SEQ ID NO: 14 - DNA, 20 bp, Artificial Sequence
  R PRIMER FOR Siah1
atcagatggggcattacagc

SEQ ID NO: 15 - DNA, 22 bp, Artificial Sequence
  F PRIMER FOR Survivin
ccactgagaacgagccagactt

SEQ ID NO: 16 - DNA, 22 bp, Artificial Sequence
  R PRIMER FOR Survivin
gacagaaaggaaagcgcaaccg

SEQ ID NO: 17 - DNA, 26 bp, Artificial Sequence
  F methylation-(M) specific primer
agttatggtaaggtaggtaggtaggc

SEQ ID NO: 18 - DNA, 24 bp, Artificial Sequence
  R methylation-(M) specific primer
aaaaaaaacttaaaaacaacgacg

SEQ ID NO: 19 - DNA, 25 bp, Artificial Sequence
  F unmethylated-(U) specific primer
tatggtaaggtaggtaggtaggtgg

SEQ ID NO: 20 - DNA, 25 bp, Artificial Sequence
  R unmethylated-(U) specific primer
aaaaaaacttaaaaacaacaacaca

SEQ ID NO: 21 - DNA, 27 bp, Artificial Sequence
  F Primer for PCR amplification
gggttgtagttatggtaaggtaggtag

SEQ ID NO: 22 - DNA, 25 bp, Artificial Sequence
  R Primer for PCR amplification
acccaaaataaataaataaaaaaaa

SEQ ID NO: 23 - DNA, 97 bp, Artificial Sequence
  Primer of shRNA′s for pan-Sept4
tgctgttgacagtgagcgcaagcacgcagtggatatagaatagtgaagccacagatgtattctatatccactgcgtgcttatgcctactgcctcgga

SEQ ID NO: 24 - DNA, 97 bp, Artificial Sequence
  Primer of shRNA′s for H5-1
tgctgttgacagtgagcgccaggaccaagccctaaaggaatagtgaagccacagatgtattcctttagggcttggtcctgttgcctactgcctcgga

SEQ ID NO: 25 - DNA, 97 bp, Artificial Sequence
  Primer of shRNA′s for H5-2
tgctgttgacagtgagcgaaaggaacggaatcgcaacaaatagtgaagccacagatgtatttgttgcgattccgttccttctgcctactgcctcgga

SEQ ID NO: 26 - DNA, 97 bp, Artificial Sequence
  Primer of shRNA′s for H5-3
tgctgttgacagtgagcgccagatgaaggagactcactaatagtgaagccacagatgtattagtgagtctccttcatctgttgcctactgcctcgga

SEQ ID NO: 27 - DNA, 97 bp, Artificial Sequence
  Primer of shRNA′s for ARTS-1
tgctgttgacagtgagcgcaagggaacaggccgagtagcatagtgaagccacagatgtatgctactcggcctgttcccttatgcctactgcctcgga

SEQ ID NO: 28 - DNA, 97 bp, Artificial Sequence
  Primer of shRNA′s for ARTS-2
tgctgttgacagtgagcgaaacaggccgagtagcaccacatagtgaagccacagatgtatgtggtgctactcggcctgttctgcctactgcctcgga

SEQ ID NO: 29 - DNA, 97 bp, Artificial Sequence
  Primer of shRNA′s for ARTS-3
tgctgttgacagtgagcgacgggtggtcactgattcctgttagtgaagccacagatgtaacaggaatcagtgaccacccgctgcctactgcctcgga

SEQ ID NO: 30 - DNA, 39 bp, Artificial Sequence
  sense primer for PCR-amplification
ggaggtaccaccatggatgacaaggagtatgtgggcttt

SEQ ID NO: 31 - DNA, 38 bp, Artificial Sequence
  Anti- sense primers for PCR-amplification
ggatctagaacatccaatggccggagcctggggaacag

SEQ ID NO: 32 - DNA, 45 bp, Artificial Sequence
  PRIMER FOR wobble mutations
ggcagggctaccacaagtggatggtcgctcattcctgttccccag

SEQ ID NO: 33 - DNA, 45 bp, Artificial Sequence
  PRIMER FOR wobble mutations
ctggggaacaggaatgagcgaccatccacttgtggtagccctgcc

SEQ ID NO: 34 - DNA, 22 bp, Artificial Sequence
  F PRIMER FOR A (-893 to -822)
cctgaccagctcagaccctact

SEQ ID NO: 35 - DNA, 21 bp, Artificial Sequence
  R PRIMER FOR A (-893 to -822)
gaaggcagatgcttggtgagt

SEQ ID NO: 36 - DNA, 22 bp, Artificial Sequence
  F PRIMER FOR B (-820 to -759)
cctagctgctgctctgaccttt

SEQ ID NO: 37 - DNA, 21 bp, Artificial Sequence
  R PRIMER FOR B (-820 to -759)
gacagatgccccagaagcttt

SEQ ID NO: 38 - DNA, 20 bp, Artificial Sequence
  F PRIMER FOR C (-651 to -589)
aaggctacaccgccctgtta

SEQ ID NO: 39 - DNA, 23 bp, Artificial Sequence
  R PRIMER FOR C (-651 to -589)
ggatcatctgctggaaggataga

SEQ ID NO: 40 - DNA, 20 bp, Artificial Sequence
  F PRIMER FOR D (-602 to -534)
ccagcagatgatccctggat

SEQ ID NO: 41 - DNA, 24 bp, Artificial Sequence
  R PRIMER FOR D (-602 to -534)
tcatgcagtaccccagattacaga

SEQ ID NO: 42 - DNA, 25 bp, Artificial Sequence
  F PRIMER FOR E (-457 to -394)
gtagtgatcagtaacttgggcatca

SEQ ID NO: 43 - DNA, 20 bp, Artificial Sequence
  R PRIMER FOR E (-457 to -394)
cgcagggattcaacaaggtt

SEQ ID NO: 44 - DNA, 21 bp, Artificial Sequence
  F PRIMER FOR F (-302 to -236)
ttctttgggaactggctcaag

SEQ ID NO: 45 - DNA, 23 bp, Artificial Sequence
  R PRIMER FOR F (-302 to -236)
tggacacctggctcttttttact

SEQ ID NO: 46 - DNA, 25 bp, Artificial Sequence
  F PRIMER FOR G (+446 to +529)
ctgggtcagcttttacctgagtaac

SEQ ID NO: 47 - DNA, 22 bp, Artificial Sequence
  R PRIMER FOR G (+446 to +529)
gcgttttccaaatgaagttggt

SEQ ID NO: 48 - DNA, 21 bp, Artificial Sequence
  F PRIMER FOR H (+708 to +842)
acaggaaccatgggaaagctt

SEQ ID NO: 49 - DNA, 26 bp, Artificial Sequence
  R PRIMER FOR H (+708 to +842)
gggttactgtttctcttcccactaca

SEQ ID NO: 50 - DNA, 22 bp, Artificial Sequence
  F PRIMER FOR I (+862 to +927)
tgcccaggtaggaagtttttga

SEQ ID NO: 51 - DNA, 23 bp, Artificial Sequence
  R PRIMER FOR I (+862 to +927)
ggcaacaatgtcaggagaagaga

SEQ ID NO: 52 - DNA, 18 bp, Artificial Sequence
  F PRIMER FOR J (+997 to +1055)
ggaagccccacagccttt

SEQ ID NO: 53 - DNA, 21 bp, Artificial Sequence
  R PRIMER FOR J (+997 to +1055)
ccccctgctgtaaacagtttg

SEQ ID NO: 54 - protein, 274 aa, Homo sapiens
MIKRFLEDTTDDGELSKFVKDFSGNASCHPPEAKTWASRPQVPEPRPQAPDLYDDDLEFRPPSRPQSSDNQQYFCAPAPLSPSARPRSPWGKLDPYDSSEDDKEYVGFATLPNQVHRKSVKKGFDFTLMVAGESGLGKSTLVNSLFLTDLYRDRKLLGAEERIMQTVEITKHAVDIEEKGVRLRLTIVDTPGFGDAVNNTECWKPVAEYIDQQFEQYFRDESGLNRKNIQDNRVHCCLYFISPFGHGYGPSLRLLAPPGAVKGTGQEHQGQGCH

SEQ ID NO: 55 - DNA, 1911 bp, Homo sapiens
cggcggtgctgcgaggtcggcgcgcacgtccgccgcgggtcgctcgggcgctgtccaggcggagccggccccgcccgggctgcagccatgatcaagcgtttcctggaggacaccacggatgatggagaactgagcaagttcgtgaaggatttctcaggaaatgcgagctgccacccaccagaggctaagacctgggcatccaggccccaagtcccggagccaaggccccaggccccggacctctatgatgatgacctggagttcagacccccctcgcggccccagtcctctgacaaccagcagtacttctgtgccccagcccctctcagcccatctgccaggccccgcagcccatggggcaagcttgatccctatgattcctctgaggatgacaaggagtatgtgggctttgcaaccctccccaaccaagtccaccgaaagtccgtgaagaaaggctttgactttaccctcatggtggcaggagagtctggcctgggcaaatccacacttgtcaatagcctcttcctcactgatctgtaccgggaccggaaacttcttggtgctgaagagaggatcatgcaaactgtggagatcactaagcatgcagtggacatagaagagaagggtgtgaggctgcggctcaccattgtggacacaccaggttttggggatgcagtcaacaacacagagtgctggaagcctgtggcagaatacattgatcagcagtttgagcagtatttccgagacgagagtggcctgaaccgaaagaacatccaagacaacagggtgcactgctgcctgtacttcatctcacccttcggccatgggtatggtccaagcctgaggctcctggcaccaccgggtgctgtcaagggaacaggccaagagcaccaggggcagggctgccactagcaggtggtcacaggttcctgttccccaggctccggccattggatgttgaattcatgaaggccctgcatcagcgggtcaacatcgtgcctatcctggctaaggcagacacactgacacctcccgaagtggaccacaagaaacgcaaaatccgggaggagattgagcattttggaatcaagatctatcaattcccagactgtgactctgatgaggatgaggacttcaaattgcaggaccaagccctaaaggaaagcatcccatttgcagtaattggcagcaacactgtagtagaggccagagggcggcgagttcggggtcgactctacccctggggcatcgtggaagtggaaaacccagggcactgcgactttgtgaagctgaggacaatgctggtacgtacccacatgcaggacctgaaggatgtgacacgggagacacattatgagaactaccgggcacagtgcatccagagcatgacccgcctggtggtgaaggaacggaatcgcaagtatgaccagaagccaggacaaagctggcagggggagatcccaagcctagccttgggtgagaccaagccctacttttgttcttctataggccctgggctcaatctaagcgggtgctggggtcctcctcgccttatcaacccttttctccctttagcaaactgactcgggaaagtggtaccgacttccccatccctgctgtcccaccagggacagatccagaaactgagaagcttatccgagagaaagatgaggagctgcggcggatgcaggagatgctacacaaaatacaaaaacagatgaaggagaactattaactggctttcagccctggatatttaaatctcctcctcttcttcctgtccatgccggcccctcccagcaccagctctgctcaggccccttcagctactgccacttcgccttacatccctgctgactgcccagagactcagaggaaataaagtttaataaatctgtaggtggctaaaaa

SEQ ID NO: 56 - protein, 634 aa, Homo sapiens
MDRSLGWQGNSVPEDRTEAGIKRFLEDTTDDGELSKFVKDFSGNASCHPPEAKTWASRPQVPEPRPQAPDLYDDDLEFRPPSRPQSSDNQQYFCAPAPLSPSARPRSPWGKLDPYDSSEDDKEYVGFATLPNQVHRKSVKKGFDFTLMVAGESGLGMDRSLGWQGNSVPEDRTEAGIKRFLEDTTDDGELSKFVKDFSGNASCHPPEAKTWASRPQVPEPRPQAPDLYDDDLEFRPPSRPQSSDNQQYFCAPAPLSPSARPRSPWGKLDPYDSSEDDKEYVGFATLPNQVHRKSVKKGFDFTLMVAGESGLGKSTLVNSLFLTDLYRDRKLLGAEERIMQTVEITKHAVDIEEKGVRLRLTIVDTPGFGDAVNNTECWKPVAEYIDQQFEQYFRDESGLNRKNIQDNRVHCCLYFISPFGHGLRPLDVEFMKALHQRVNIVPILAKADTLTPPEVDHKKRKIREEIEHFGIKIYQFPDCDSDEDEDFKLQDQALKESIPFAVIGSNTVVEARGRRVRGRLYPWGIVEVENPGHCDFVKLRTMLVRTHMQDLKDVTRETHYENYRAQCIQSMTRLVVKERNRNKLTRESGTDFPIPAVPPGTDPETEKLIREKDEELRRMQEMLHKIQKQMKENY

SEQ ID NO: 57 - DNA, 1767 bp, Homo sapiens
gaaaggagcaagccaggaagccagacaacaacagcatcaaaacaaggctgtttctgtgtgtgaggaactttgcctgggagataaaattagacctagagctttctgacagggagtctgaagcgtgggacatggaccgttcactgggatggcaagggaattctgtccctgaggacaggactgaagctgggatcaagcgtttcctggaggacaccacggatgatggagaactgagcaagttcgtgaaggatttctcaggaaatgcgagctgccacccaccagaggctaagacctgggcatccaggccccaagtcccggagccaaggccccaggccccggacctctatgatgatgacctggagttcagacccccctcgcggccccagtcctctgacaaccagcagtacttctgtgccccagcccctctcagcccatctgccaggccccgcagcccatggggcaagcttgatccctatgattcctctgaggatgacaaggagtatgtgggctttgcaaccctccccaaccaagtccaccgaaagtccgtgaagaaaggctttgactttaccctcatggtggcaggagagtctggcctgggcaaatccacacttgtcaatagcctcttcctcactgatctgtaccgggaccggaaacttcttggtgctgaagagaggatcatgcaaactgtggagatcactaagcatgcagtggacatagaagagaagggtgtgaggctgcggctcaccattgtggacacaccaggttttggggatgcagtcaacaacacagagtgctggaagcctgtggcagaatacattgatcagcagtttgagcagtatttccgagacgagagtggcctgaaccgaaagaacatccaagacaacagggtgcactgctgcctgtacttcatctcacccttcggccatgggctccggccattggatgttgaattcatgaaggccctgcatcagcgggtcaacatcgtgcctatcctggctaaggcagacacactgacacctcccgaagtggaccacaagaaacgcaaaatccgggaggagattgagcattttggaatcaagatctatcaattcccagactgtgactctgatgaggatgaggacttcaaattgcaggaccaagccctaaaggaaagcatcccatttgcagtaattggcagcaacactgtagtagaggccagagggcggcgagttcggggtcgactctacccctggggcatcgtggaagtggaaaacccagggcactgcgactttgtgaagctgaggacaatgctggtacgtacccacatgcaggacctgaaggatgtgacacgggagacacattatgagaactaccgggcacagtgcatccagagcatgacccgcctggtggtgaaggaacggaatcgcaacaaactgactcgggaaagtggtaccgacttccccatccctgctgtcccaccagggacagatccagaaactgagaagcttatccgagagaaagatgaggagctgcggcggatgcaggagatgctacacaaaatacaaaaacagatgaaggagaactattaactggctttcagccctggatatttaaatctcctcctcttcttcctgtccatgccggcccctcccagcaccagctctgctcaggccccttcagctactgccacttcgccttacatccctgctgactgcccagagactcagaggaaataaagtttaataaatctgtaggtggctaaaaaaaaaaaaaaaaaaaaaaaaaaaaaaa

(19) United States
(12) Patent Application Publication
(10) Pub. No.: US 2016/0047001 A1
(43) Pub. Date: Feb. 18, 2016

(54) Sept4/ARTS AS A TUMOR SUPPRESSOR IN THE DIAGNOSIS, PROGNOSIS AND TREATMENT OF HEPATIC DISORDERS

(71) Applicants: CARMEL-HAIFA UNIVERSITY ECONOMIC CORPORATION LTD., Haifa (IL); THE ROCKEFELLER UNIVERSITY, New York, NY (US)

(72) Inventors: Sarit LARISCH, Zichron Yaakov (IL); Hermann STELLER, East Burke, VT (US)

(73) Assignees: CARMEL-HAIFA UNIVERSITY ECONOMIC CORPORATION LTD., Haifa (IL); THE ROCKEFELLER UNIVERSITY, New York, NY (US)

(21) Appl. No.: 14/783,129

(22) PCT Filed: Apr. 7, 2014
(86) PCT No.: PCT/IL2014/050335

§ 371 Date: Oct. 8, 2015

Related U.S. Application Data
(60) Provisional application No. 61/809,587, filed on Apr. 8, 2013.

(51) Int. Cl. C12Q 1/68; A61K 31/185; A61K 31/53

(52) U.S. Cl. CPC C12Q 1/6886; A61K 31/53; A61K 31/185; C12Q 2600/118; C12Q 2600/154

(57) ABSTRACT

The invention relates to methods for the diagnosis and prognosis of hepatic disorder and associated pathologies as well as of a solid proliferative disorder in a mammalian subject. More specifically, the methods of the invention are based on determining the expression, methylation of ARTS as well as histone trimethylation. The invention further provides therapeutic methods for treating said disorders.

### FIELD OF THE INVENTION

The invention relates to diagnosis and prognosis of hepatic disorders and solid proliferative disorders. More particularly, the invention provides the diagnosis, prognosis and treatment of solid tumors and specifically of hepatic disorders using ARTS as a bio-marker.

### BACKGROUND REFERENCES

References considered being relevant as background to the presently disclosed
1. Hanahan D, Weinberg R A (2011) The hallmarks of cancer: the next generation. Cell 144:646-674.
2. Kitada S, Pedersen I M, Schimmer A D, Reed J C (2002) Dysregulation of apoptosis genes in hematopoietic malignancies. Oncogene 21:3459-3474.
3. Lowe S W, Lin A W (2002) Apoptosis in cancer. Carcinogenesis 2000 21: 485-495.
4. Reed J C, Pellecchia M (2005) Apoptosis-based therapies for hematologic malignancies. Blood 106:408-416.
5. Salvesen G S, Duckett C S (2002) IAP Proteins: blocking the road to death's door. Nat Rev Mol Cell Biol 3:401-410.
6. Vaux D L, Silke J (2005) IAPB, RINGS and ubiquitylation. Nat Rev Mol Cell Biol 6:287-297.
7. Schile A J, Garcia-Fernandez M, Steller H (2008) Regulation of apoptosis by XIAP ubiquitin-ligase activity. Genes Dev 22:2256-2266.
8. Yang Y, Fang S, Jensen J P, Weissman A M, Ashwell J D (2000) Ubiquitin protein ligase activity of IAPB and their degradation in proteasomes in response to apoptotic stimuli. Science 288:874-877.
9. Li L, et al. (2004) A small molecule Smac mimic potentiates TRAIL- and TNFα-mediated cell death. Science 305:1471-1474.
10. Petersen S L, et al. (2007) Autocrine TNFα signaling renders human cancer cells susceptible to Smac-mimetic-induced apoptosis. Cancer Cell 12:445-456.
11. Schimmer A D, et al. (2004) Small-molecule antagonists of apoptosis suppressor XIAP exhibit broad antitumor activity. Cancer Cell 5:25-35.
12. Varfolomeev E, et al. (2007) IAP antagonists induce autoubiquitination of c-IAPB, NF-κB activation, and TNFα-dependent apoptosis. Cell 131:669-681.
13. Vince J E, et al. (2007) IAP antagonists target cIAP1 to induce TNFα-dependent apoptosis. Cell 131:682-693.
14. Du C, Fang M, Li Y, Li L, Wang X (2000) Smac, a mitochondrial protein that promotes cytochrome c-dependent activation by eliminating IAP inhibition. Cell 102:33-42.
15. Suzuki Y, et al. (2001) A serine protease HtrA2, is released from the mitochondria and interacters with XIAP, inducing cell death. Mol Cell Biol 8:613-621.
16. Verhagen A M, et al. (2000) Identification of DIABLO, a mammalian protein that promotes apoptosis by binding to and antagonizing IAP proteins. Cell 102:43-53.
17. Jones J M, et al. (2003) Loss of Omi mitochondrial protease activity causes the neuromuscular disorder of mnd2 mutant mice. Nature 425:721-727.
18. Martins L M, et al. (2004) Neuroprotective role of the reaper-related serine protease HtraA2/Omi revealed by targeted deletion in mice. Mol Cell Biol 24:9848-9862.
19. Okada H, et al. (2002) Generation and characterization of Smac/Diablo-deficient mice. Mol Cell Biol 22:3509-3517.
20. Larisch S, et al. (2000) A novel mitochondrial septin-like protein, ARTS, mediates apoptosis dependent on its P-loop motif. Nat Cell Biol 2:915-921.
21. Gottfried Y, Rotem A, Lotan R, Steller H, Larisch S (2004) The mitochondrial ARTS protein promotes apoptosis through targeting XIAP. EMBO J 23:1627-1635.
22. Garrison J B, et al. (2011) ARTS and Siah collaborate in a pathway for XIAP degradation. Mol Cell 41:107-116.
23. Elhasid R, et al. (2004) Mitochondrial pro-apoptotic ARTS protein is lost in the majority of acute lymphoblastic leukemia patients. Oncogene 23:5468-5475.
24. Garcia-Fernandez M, et al. (2010) Sept4/ARTS is required for stem cell apoptosis and tumor suppression. Genes Dev 24:2282-2293.
25. Sherman M (2010) Hepatocellular carcinoma: epidemiology, surveillance, and diagnosis. Sem Liver Dis 30:3-16.
26. Kissel H, et al. (2005) The Sept4 septin locus is required for sperm terminal differentiation in mice. Dev Cell 8:353-364.
27. Goldberg et al. (2010), Cell 140: 678.
28. Ikeguchi M, Ueda T, Sakatani T, Hirooka Y, Kaibara N (2002) Expression of survivin messenger RNA correlates with poor prognosis in patients with hepatocellular carcinoma. Diagn Mol Pathol 11:33-40.
29. Jones P A, Baylin, S B (2007) The epigenomics of cancer. Cell 128:683-692.
30. Zender L, et al. (2006) Identification of oncogenes in liver cancer using an integrative oncogenomic approach. Cell 125:1253-1267.
31. Chi P, Allis C D, Wang G G (2010) Covalent histone modifications—miswritten, misinterpreted and mis-erased in human cancers. Nat Rev Cancer 10:457-469.
32. Liang G, et al. (2004) Distinct localization of histone H3 acetylation and H3-K4 methylation to the transcription start site in the human genome. Proc Natl Acad Sci 101:7357-7362.
33. Kondo Y, et al. (2008) Gene silencing in cancer by histone H3 lysine 27 trimethylation independent of promoter DNA methylation. Nature Genetics 40:741-750.
34. Vesselinovitch S D, Mihailovich N. (1983) Kinetics of diethylnitrosmine hepatocarcinogenesis in the infant mouse. Cancer Res 43:4263-4259.
35. Murakami H, et al. (1993) Transgenic mouse model for synergistic effects of nuclear oncogenes and growth factors in tumorigenesis: Interaction of c-myc and transforming growth factor-α in hepatic oncogenesis. Cancer Res 53:1719-1723.
36. Fuchs and Steller (2011). Programmed cell death in animal development. Cell 147, 742-58.

Acknowledgement of the above references herein is not to be inferred as meaning that these are in any way relevant to the patentability of the presently disclosed subject matter.

### BACKGROUND OF THE INVENTION

One important role of apoptosis is to eliminate dangerous cells, such as cancer cells (1, 15, and 36). Evidence for a critical role of apoptosis as a tumor-suppressor mechanism has largely come from work on hematopoietic malignancies (2-4), but its precise role in restricting the formation of solid tumors is less clear. In particular, only very few cell death genes have been shown to function as tumor suppressors in solid tumors. Therefore, much remains to be learned about the precise role of apoptosis in restricting solid tumors.

Inhibitor of apoptosis (IAP) proteins directly bind and inhibit caspase proteases (5, 6), the executioners of apoptosis. Many IAPB also possess E3 ubiquitin-ligase activity that confers them with the ability to ubiquitinate key target cell death proteins, including caspases and IAPB themselves for proteasome-mediated degradation (5-8). Significantly, because IAPB are frequently over-expressed in human tumors, they have become important targets in the development of anti-cancer drugs (9-14).

During the induction of apoptosis, endogenous proteins termed IAP-antagonists negatively regulate IAP function. Molecular genetic studies of apoptosis in Drosophila originally revealed the central importance of the IAP antagonists reaper, grim, and head involution defective (hid) for the induction of apoptosis in response to many different cell death signals (15). In mammals, IAP-antagonists like Smac/DIABLO (Smac) and Omi/HtrA2 (Omi) reside within mitochondria in living cells and are released into the cytosol at the onset of apoptosis (14-16). Inactivation of either Smac/DIABLO, Omi/HtrA2, or both together in double-mutant mice does not lead to increased resistance towards cell death or increased tumor formation (17-19). Therefore, the physiological role of these proteins remains to be established.

ARTS/Sept4_i2 (ARTS), an isoform encoded by the Septin-4 (Sept4) gene, is another mammalian IAP-antagonist (20, 21). ARTS stimulates degradation of X-linked Inhibitor of Apoptosis Protein (XIAP) by promoting complex formation between XIAP and the E3-ligase protein Siah1 (22). Loss of ARTS function has been associated with leukemia (23), and Sept4/ARTS-deficient mice display susceptibility to hematopoietic malignancy (24).

Thus, there is need for biological markers for early diagnosis, prognosis and monitoring of processes leading to solid malignancies. Markers involved in aberrant apoptotic processes are highly valuable as prognostic and diagnostic tools and for personalized medicine.

### SUMMARY OF THE INVENTION

A first aspect of the invention relates to a method for the diagnosis and prognosis of a hepatic disorder and associated pathologies as well as of a solid proliferative disorder in a mammalian subject. More specifically, the method of the invention comprises the steps of: (a), determining in at least one biological sample of the examined subject at least one of: (i) the level of expression of Apoptosis Related Protein in the TGF-beta Signaling Pathway (ARTS) and optionally of at least one of Survivin and α-fetoprotein (AFP) to obtain an expression value; (ii) Sept4/ARTS methylation level or (iii) the level of Histone 3 trimethylation at at least one of lysine 4, lysine 9 and lysine 27 to obtain a trimethylation value of histone H3 at said lysine residues.

The present invention further provides the use of ARTS as an early marker for detecting early stages of a malignant process, or alternatively, early detection of relapse of patients treated with a certain therapeutic agent. In yet other embodiments, the invention provides a method for assessing responsiveness of a mammalian subject to treatment with a specific therapeutic agent or evaluating the efficacy of treatment on a subject.

In yet another aspect, the invention provides a method for treating, preventing, ameliorating or delaying the onset of a hepatic disorder and associated pathologies or of a solid proliferative disorder in a subject in need thereof. The therapeutic method of the invention combines the diagnostic steps as described herein above and further requires the step of administering a therapeutically effective amount of at least one chromatin modifying drug or ARTS or any fragment, peptide, analogues and derivatives thereof or any composition comprising the same, to a subject displaying at least one of (i) a negative expression value of ARTS and optionally, a positive expression value of at least one of Survivin and AFP; (ii) a positive value of Sept4/ARTS TSS methylation; and (iii) a negative trimethylation value of histone H3 at lysine 4 or a positive value at lysine 9 or lysine 27 as determined in step (b).

In yet a further aspect, the invention provides a kit comprising means for performing at least two of:

(a) determining the level of expression of ARTS and optionally of at least one of Survivin and AFP in a biological sample;
(b) determining the level of Sept4/ARTS methylation of the CpG islands at the TSS in a biological sample; and
(c) determining the level of Histone 3 trimethylation of at least one of lysine 4, lysine 9 and lysine 27 in a biological sample.

In certain embodiments the kit of the invention may be also used for therapeutic purposes.

These and other aspects of the invention will become apparent by the hand of the following drawing.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1G. Expression of ARTS is frequently lost in liver cancer

FIG. 1A. qRT-PCR showing ARTS mRNA levels normalized to β-actin in primary tumors from liver cancer patients (1-40). While patients 1-4 showed normal expression (˜1.0-fold), and patients 5-8 displayed higher abundance (*45,000, **14.2, ***2.5, and 1.4-fold), the majority of patients (9-40) displayed a reduction (0.9-fold or less). Wilcoxon signed rank and rank sum tests indicated a statistical significance between patients and normal controls (p<0.001) and (p=0.004), respectively. Ct-values for ARTS and β-actin are reported in Table 1.

FIGS. 1B and 1C. levels of Survivin and AFP mRNA. Values are shown as fold-difference compared to non-malignant tissue, as in FIG. 1A above. *, **, and # indicate increases of 45-to-200, 800-to-900, and >8000-fold, respectively.

FIG. 1D. Semi-quantitative RT-PCR showing ARTS mRNA levels in HCC cell-lines are reduced in comparison to normal adult hepatocytes (upper panel).

FIG. 1E. Quantification of ARTS expression, normalized to β-actin mRNA, was performed by densiometric analysis (Alpha-Innotech) (lower panel).

FIG. 1F. ARTS expression, determined by qPCR and normalized to β-actin is shown relative to expression in THLE-2 cells. Values are the mean±standard deviation of three independent experiments.

FIG. 1G. RT-PCR showing the levels of α-fetoprotein (AFP) and other indicated mRNAs.

FIG. 2A-2E. Epigenetic regulation of the Sept4/ARTS promoter in HCC

FIG. 2A. The human Sept4 genomic locus (located in chromosome 17) encodes Sept4-iso forms H5 (Sept4_i1) and ARTS (Sept4_i2). Sept/ARTS contains an alternative proximal promoter and transcriptional start site (bent arrow).

FIG. 2B. A high density of CpG dinucleotides near the ARTS TSS (+1, indicated by arrow) forms a predicted CpG island. Eighty-four CpGs reside within 500 base-pairs flanking the ARTS TSS.

FIG. 2C. RT-PCR showing upregulation of ARTS mRNA following exposure to AZA (10 μM), TSA (1 μM), or co-treatment of both in the indicated cell-lines.

FIG. 2D. Results of methylation-specfic PCR. Methylated (M)- and unmethylated (U) primer sets correspond to Sept4/ARTS genomic intervals ⁺84 to ⁺284 (M) or ⁺87 to ⁺283 (U). In vitro methylated (+IVM) DNA served as a positive control.

FIG. 2E. Human Sept4/H5 (Sept4_i1) does not contain a CpG-island in its proximal promoter, Depiction of the genomic region of human Sept4/H5 (Sept4_i1) (GeneBank NM_{—}004574.2, it should be noted that the nucleic acid sequence is denoted by SEQ ID NO. 57, and the amino acid sequence is denoted by SEQ ID NO. 56), from ⁻1000 to ⁺1000 nucleotides (nt) with respect to the transcriptional start site (TSS). Based on the GC percentage (y-axis) and relative lack of CpG dinucleotides, the Sept4/H5 proximal promoter lacks any apparent CpG-island, the number of nucleotides with respect to the TSS (bent arrow) is shown in the x-axis. Each CpG dinucleotide, represented by a vertical bar at the bottom, appears dispersed within this genomic region.

FIG. 3A-3G. Repressive epigenetic signature of the Sept4/ARTS promoter in HCC

FIGS. 3A and 3B. Hypermethylation of the Sept4/ARTS promoter in HCC cell-lines based on bisulfite sequencing. The profiles show the methylation status of each cytosine in the CpGs between nucleotides +249 and +443 near the ARTS TSS (underlined in red in FIG. 3A). In FIG. 3B, each row reflects an individually cloned allele. Open and filled boxes represent an unmethylated or methylated CpG, respectively.

FIGS. 3C and 3D. Frequency of CpG-methylation within Sept4/ARTS between positions +109 through +443. Frequency (%) is calculated from a minimum of 15 individually cloned alleles.

FIGS. 3E-3G. ChIP results showing trimethylation of H3K4 (H3K4me3), H3K9 (H3K9me3), and H3K27 (H3K27me3) across the Sept4/ARTS TSS, as shown.

FIG. 4A-4L. Loss of Sept4 leads to accelerated HCC in chemically-induced and genetic liver cancer models

FIG. 4A. Incidence of macroscopic liver lesions in mice following DEN/PB treatment. Each cohort was comprised of at least 10 mice. *P<0.0001 for Sept4^{-/-} in comparison to Sept4^{+/+} at 10 months of age.

FIG. 4B. Incidence of HCC in animals following DEN/PB treatment. *P=0.06, **P=0.002 for Sept4^{+/-} and Sept4^{-/-} respectively, in comparison to Sept4^{+/+}.

FIG. 4C. Total number of macroscopic lesions per liver in 10-month old mice shown in FIGS. 4A and 4B above.

FIG. 4D. Liver-to-whole body weight percentages of 10-month old mice and of non-DEN/PB-treated mice are shown.

FIG. 4E. Representative DEN/PB-affected livers from Sept4^{+/+} (+/+) and Sept4^{-/-} (−/−) mice.

FIG. 4F. Serum ALT levels from 10-month old mice. Values are the mean±standard deviation. One Sept4^{+/+} mouse appeared to be an outlier with ALT value 1875 IU/L, hence this mouse was excluded from calculation of the mean.

FIG. 4G. Histology of HCC from DEN/PB-treated Sept4^{-/-} mice. Lesions with solid or sinusoidal cord-like pattern, left and right, respectively.

FIG. 4H. A representative HCC from Sept4^{-/-} mice shown by hematoxylin and eosin staining (H&E) and by immunohistochemistry (IHC) detecting Ki-67, active caspase-3, and TUNEL. The panels reflect serial sections of a single carcinoma. Control IHC experiments were performed in parallel on different tissues to verify antibody binding (shown in FIG. 4K).

FIG. 4I. Immunoblots showing levels of the indicated proteins (at left) in DEN/PB-induced tumors (T) or control, non-tumor (N) adjacent parenchyma of 10 month old Sept4^{-/-} mice.

FIG. 4J. Incidence of liver cancer in Sept4^{+/+} and Sept4^{-/-} mice on the transgenic Albumin-Myc background. Mice were 12 to 15 months old.

FIG. 4K. Accelerated DEN-induced hepatocarcinogenesis in Sept4-deficient mice. Magnetic resonance imaging (MRI) detected tumor masses in Sept4-deficient mice following DEN-induced hepatocarcinogenesis in 7- and 8-month old mice. Cross-sectional MRIs of representative DEN-treated Sept4^{+/+} mice, shown on the left side, fail to show any detectable lesions. In contrast, Sept4^{+/-} mice displayed susceptibility towards HCC as demonstrated by formation of masses observed by MRI. Large lesions were seen, as indicated by asterisk (*).

FIG. 4L. Immunohistochemistry (IHC) staining controls. Control immunohistochemical staining detecting active-caspase-3, Ki-67, or TUNEL in lymphoid tissues were performed in parallel to IHCs shown in FIG. 4H to confirm antibody binding activity.

FIG. 5A-5I. Knockdown of ARTS, but not Sept4/H5, promotes oncogenesis in hepatoblasts

FIG. 5A. IHC showing A6-positive cells within a DEN/PB-triggered carcinoma region (T). In adjacent non-tumor (NT) tissue, A6-positivity appeared restricted to bile duct epithelium.

FIG. 5B. Western blot showing knockdown by ARTS-targeting hairpins. Hairpins targeting specifically ARTS (sh-ARTS-1, -2, and -3) or all Sept4-isoforms (sh-panSept4) were capable of reducing ARTS levels in NIH3T3 fibroblasts.

FIG. 5C. Cell death of PHM-1 hepatoblast lines, following exposure to cytotoxic treatments (TNFα; 100 U/ml) plus cycloheximide (CHX; 10 ug/ml), agonistic anti-Fas antibody Jo-2 (3 ug/ml) plus CHX (10 ug/ml), or serum deprivation. Values are the mean±standard deviation of three independent experiments.

FIG. 5D. Growth of PHM-1 hepatoblasts following their sub-cutaneous transplantation into host mice. Values are the mean±standard deviation of four independent experiments. P-values: *P=0.04, **P=0.03, and ***P=0.01.

FIG. 5E. Representative tumor growth promoted by ARTS-knockdown (shARTS) compared to non-silencing control (Empty).

FIG. 5F. Tumorigenesis promoted by ARTS-knockdown (Ctrl) could be partially rescued by expression of a knockdown resistant ARTS cDNA (wob).

FIG. 5G. Hematoxylin and eosin (H & E) staining showing oval cells (at arrows) found in a HCC of a DEN-PB-treated Sept4^{-/-} mouse.

FIG. 5H. Neoplastic cells in a pre-malignant liver tumor of a DEN/PB-treated Sept4^{-/-} mouse stains Antigen-6 (A6)-positive by immunohistochemistry. The surrounding normal parenchyma does not.

FIG. 5I. A pan-Sept4- and Sept4/H5-specific short-hairpins promote efficient knowndown. Western blot showing highly effective Sept4/H45 knockdown by a panSept4 and Sept4/H5 specific hairpins. NIH3T3 fibroblasts stably expressing Flag-epitope tagged Sept4/H5 (Sept4/H5-Flag) were transduced with retroviruses harboring the indicated shRNAs, a non-silencing control, or left un-infected Immunoblotting was performed with anti-Flag antibody to detect Sept4/H5-Flag expression (red arrow).

### DETAILED DESCRIPTION OF THE INVENTION

Predicting the onset of a disease at an early stage is highly valuable and clinically desired specifically for diseases that are often detected at advanced stages. Further, adjusting suitable treatment protocols is appreciated in view of the fact that a large number of treatment protocols are often associated with some extent of undesired side effects. Thus, providing tools that assist in predicting early diagnosis of a disease or predicting response of a patient to a treatment protocol at early stages after initiation of treatment and/or throughout or after a treatment period are highly important and may avoid inadequate treatments and reduce unnecessary side effects.

In addition, identifying breakthrough points throughout the disease and even after remission can asses in predicting the probability of a disease relapse, which has proved to be one of the key for successful treatment of patients.

Thus, there is a critical need for reliable predictors that will provide detection of the presence of disease at early stages and gaudiness and identification of treatment success and failure, breakthrough point and predict inadequate treatments.

In the present invention, the inventors have studied the expression of two major protein isoforms encoded by the Sept4 gene, the non-apoptotic protein, H5 or Sept4_i1 and the pro-apoptotic protein, Sept4_i2 or ARTS.

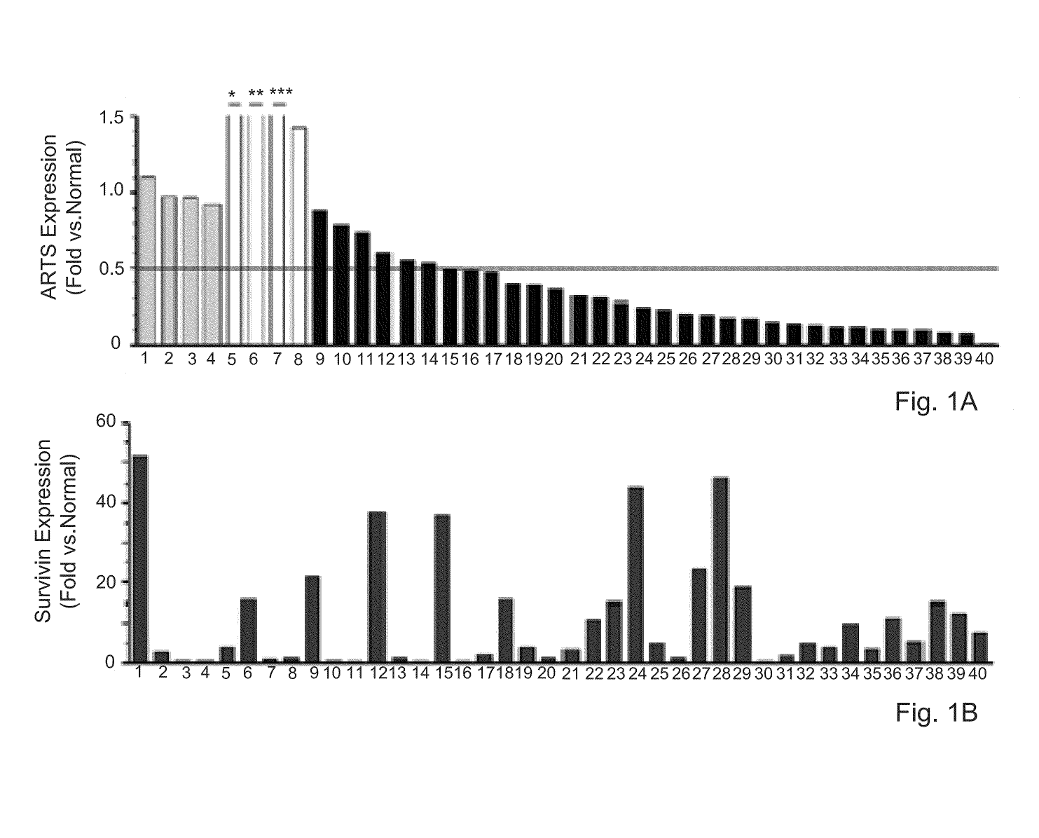

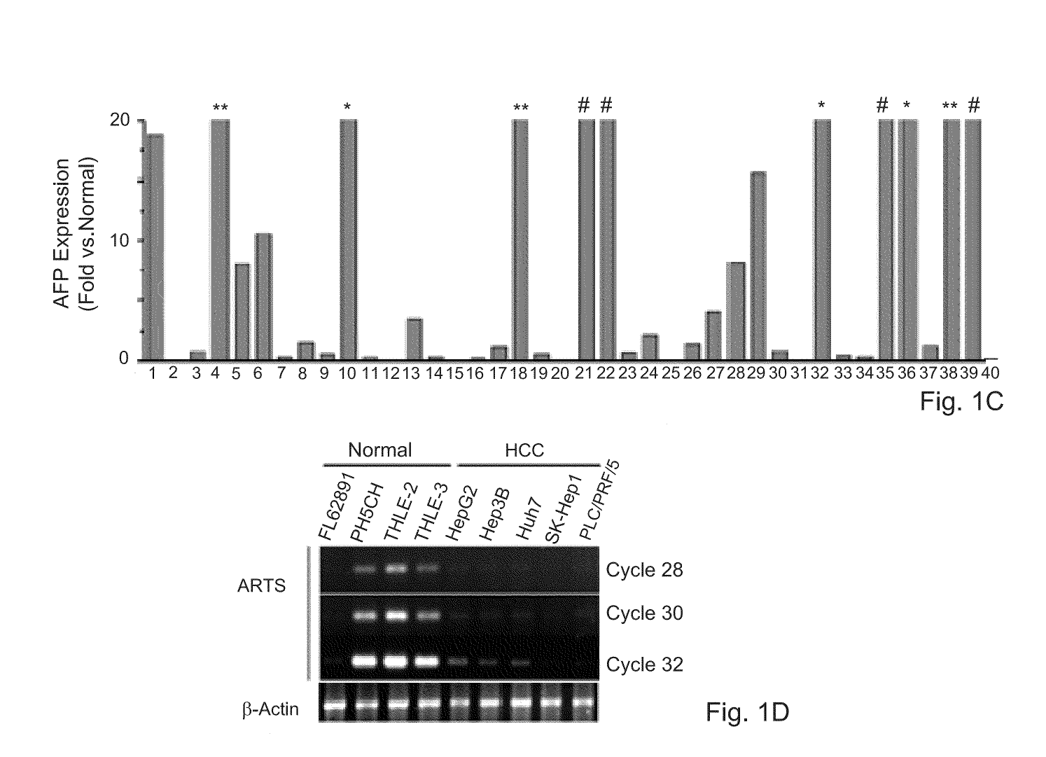

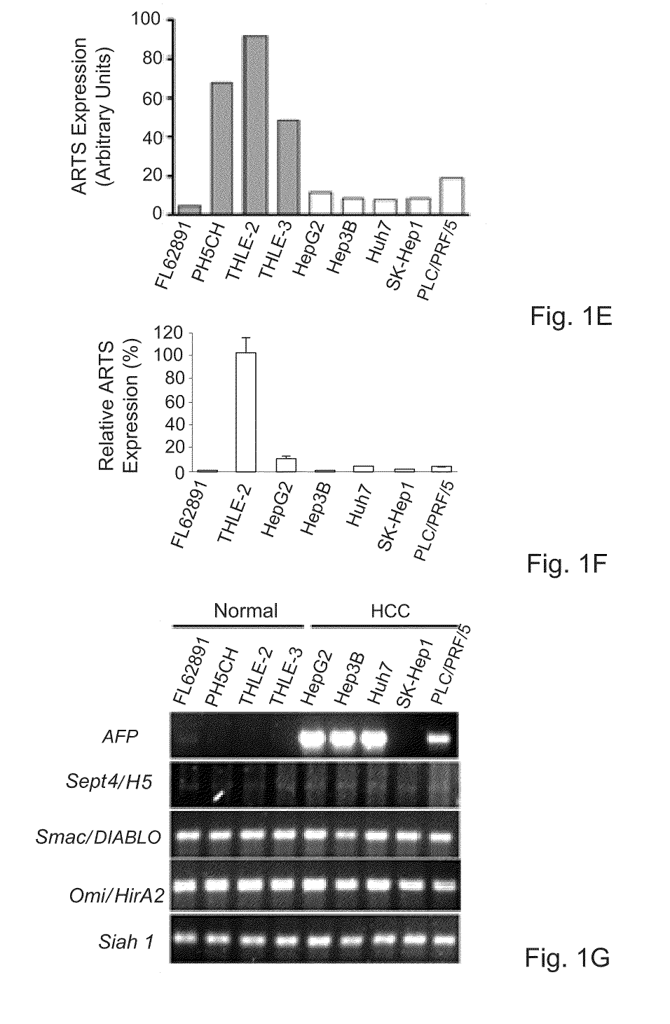

The inventors have surprisingly found that the expression of the pro-apoptotic protein ARTS, but not of the non-apoptotic protein, H5 was dramatically reduced in samples derived from HCC (hepatocellular carcinoma) patients (FIG. 1).

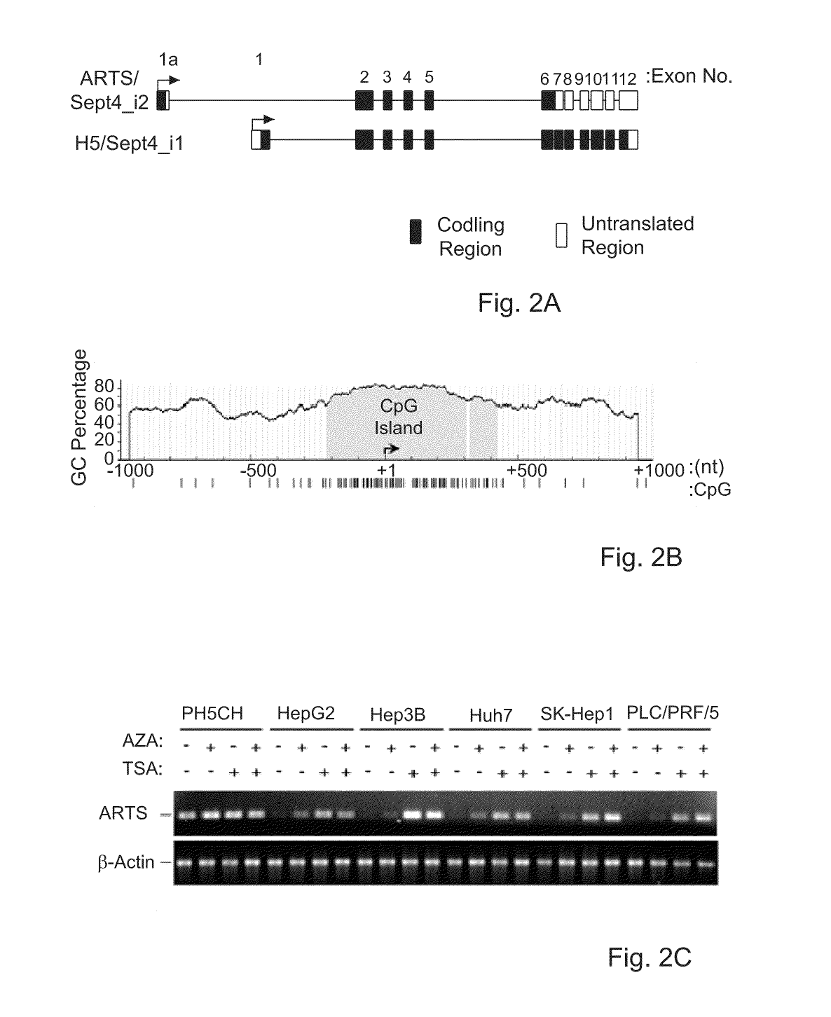

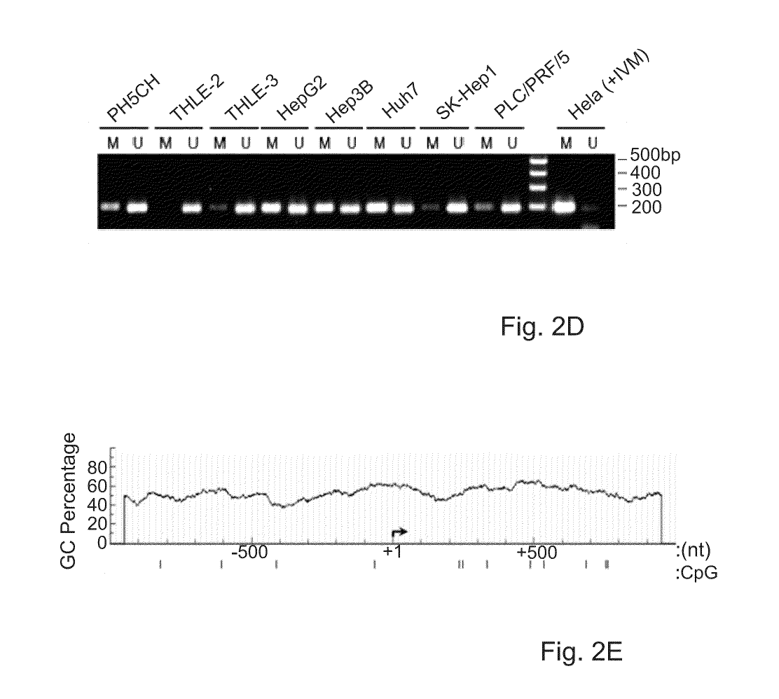

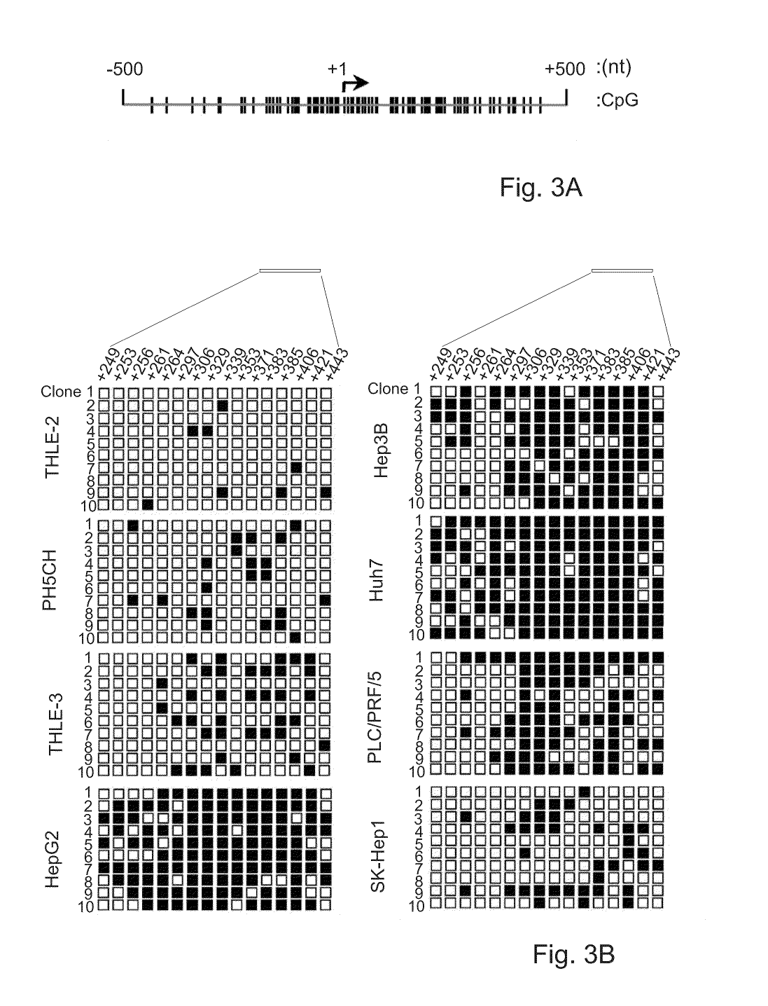

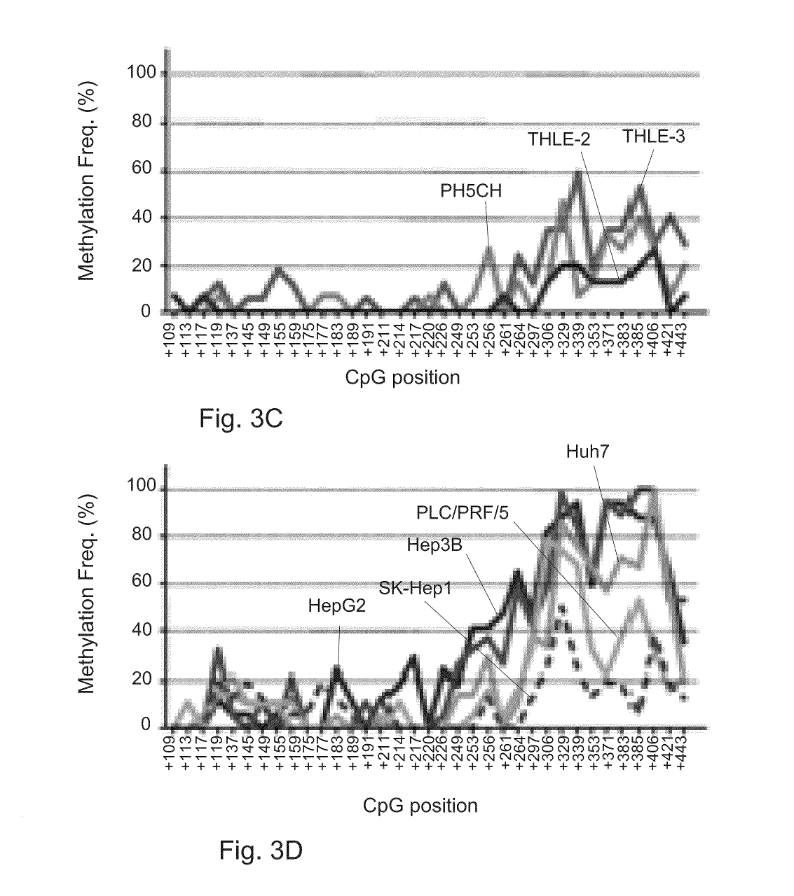

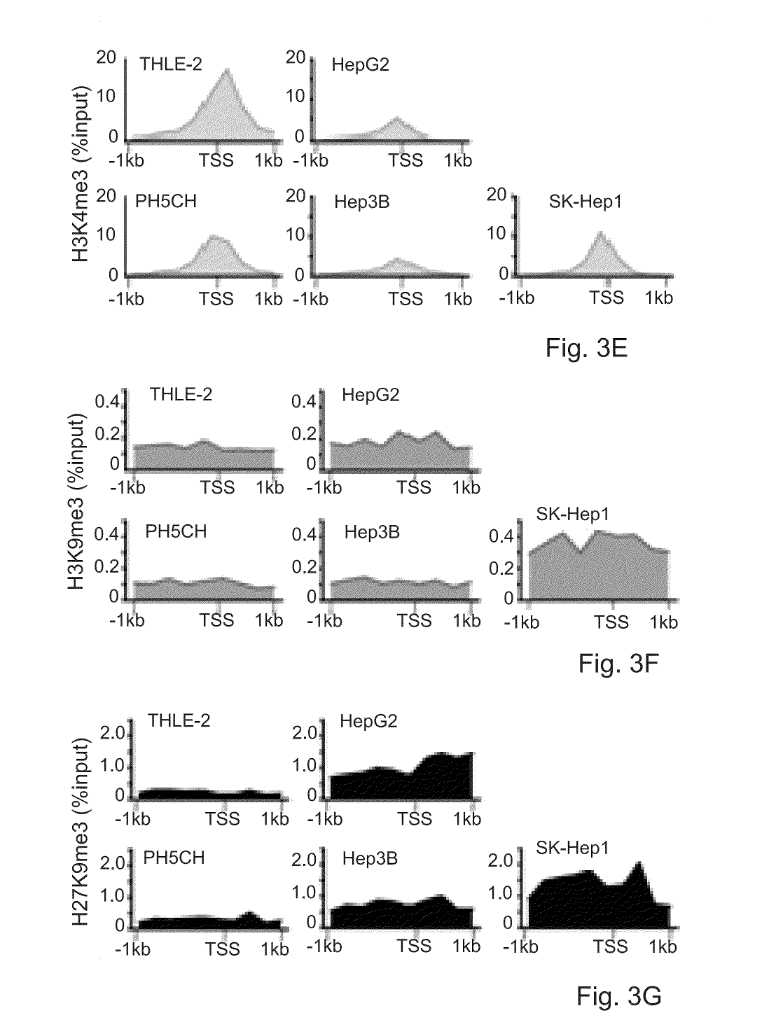

As demonstrated in FIGS. 2 and 3, the inventors have further found that the reduction in ARTS levels may be attributed to silencing inactivation of the ARTS-specific promoter by epigenetic regulation through DNA hypermethylation and/or to an induction of abnormal, repressive histone methylation signatures (FIGS. 3E-G).

The inventors have therefore concluded that determining and monitoring the expression or the epigenetic parameters involved in Sept4_i2 or ARTS expression, as detailed herein may be suitable for predicting the presence of a disease and/or assessing and monitoring response to treatment of a patient suffering from a hepatic pathological condition, specifically, HCC or any other solid tumor.

Thus, a first aspect of the invention relates to a method for the diagnosis and prognosis of a hepatic disorder and associated pathologies. In yet another embodiment, the invention relates to the diagnosis and prognosis of a solid proliferative disorder in a mammalian subject. More specifically, the method of the invention comprises the steps of: First in step (a), determining in at least one biological sample of the examined subject at least one of:

(i) the level of expression of Apoptosis Related Protein in the TGF-beta Signaling Pathway (ARTS) and optionally of at least one of Survivin and α-fetoprotein (AFP) to obtain an expression value.
(ii) Sept4/ARTS methylation level of the CpG islands at the transcription start site (TSS) to obtain a value of Sept4/ARTS TSS methylation.
(iii) the level of Histone 3 trimethylation at at least one of lysine 4, lysine 9 and lysine 27 to obtain a trimethylation value of histone H3 at said lysine residues.

The next step (b), involves determining at least one of:

(i) if the expression value of ARTS obtained in step (a i) is any one of, positive or negative with respect to a predetermined standard expression value of ARTS or to the expression value of ARTS in a control sample. In an optionally embodiment, this step involves in addition to ARTS expression, determining if the expression value of at least one of Survivin and AFP is any one of, positive or negative with respect to a predetermined standard expression value of at least one of Survivin and AFP or to the expression value of at least one of Survivin and AFP in a control sample.
(ii) if the value of Sept4/ARTS TSS methylation obtained in step (a ii) is any one of, positive or negative with respect to a predetermined standard Sept4/ARTS TSS methylation or the Sept4/ARTS TSS methylation value in a control sample.
(iii) if trimethylation value of histone H3 at said lysine residues obtained in step (a iii) is any one of, positive or negative with respect to a predetermined standard trimethylation value of histone H3 or to the trimethylation value in a control sample.

It should be noted that in certain embodiments, wherein at least one of (i) a negative expression value of ARTS and optionally, a positive expression value of at least one of Survivin and AFP; (ii) a positive value of Sept4/ARTS TSS methylation; and (iii) a negative trimethylation value of histone H3 at lysine 4 or a positive value at lysine 9 or lysine 27, indicates that the examined subject is suffering from a hepatic disorder and associated pathologies. Alternatively, the tested subject may be a subject suffering from a solid proliferative disorder.

As noted above, the method of the invention is based on the use of ARTS as a biomarker for diagnosis and prognosis of hepatic disorders and solid tumors. As used herein the term “ARTS” (apoptosis-related protein in the TGF-β signaling pathway) denotes a septin-like mitochondrial protein derived from alternative splicing of the H5/PNUTL2/′hCDCrel2a/2b gene. ARTS acts as a tumor suppressor protein that functions as an antagonist of XIAP and thereby promotes apoptosis.

It should be appreciated that in certain embodiments, as used herein in the specification and in the claim section below, ARTS protein refers to the human ARTS. More specifically, in certain embodiments the ARTS protein comprises an amino acid sequence of 274 amino acid residues as denoted by GenBank Accession No. AF176379. In some specific embodiments, the ARTS protein comprises the amino acid sequence as denoted by SEQ ID NO:54. In some other embodiments, human ARTS protein is encoded by a nucleic acid sequence provided herein below by SEQ ID NO:55.

As demonstrated in FIG. 1, the inventors provide an indication of early HCC, even in cases where Survivin and AFP, that are widely accepted biomarkers for HCC, fail to coincide with malignancy. Thus, the combined use of ARTS and at least one of Survivin and AFP as biomarkers for HCC is further provided by the invention. As used herein “survivin”, also known as baculoviral inhibitor of apoptosis repeat-containing 5 or MRCS, is a member of the inhibitors of apoptosis (IAP) family. The survivin protein functions to inhibit caspase activation, thereby leading to negative regulation of apoptosis.

As used herein “Alpha-Fetal Protein” (AFP, α-fetoprotein also known as alpha-1-fetoprotein or alpha-fetoglobulin) is a major plasma protein.

For simplicity, the three measurements performed in step (a) of the method of the invention are collectively referred to herein as “examined parameters”. It should be noted that as used herein the examined parameters denotes at least one, or at least two of or all measurements of:

(i) the level of expression of ARTS and optionally of at least one of Survivin and AFP to obtain an expression value;
(ii) Sept4/ARTS methylation level of the CpG islands at the transcription start site (TSS) to obtain a value of Sept4/ARTS TSS methylation, and
(iii) the level of Histone 3 trimethylation at at least one of lysine 4, lysine 9 and lysine 27 to obtain a trimethylation value of histone H3 at said lysine residues.

Specific embodiments relate to measurement of the level of expression according to (i) and the methylation level according to (ii). In yet another embodiment, the method comprises measuring the level of expression according to (i) and the Histone 3 trimethylation according to (iii). In another embodiment, the method of the invention involves measuring the methylation level according to (ii) and the Histone 3 trimethylation according to (iii). In still a further embodiment, step (a) involves measuring the level of expression according to (i), the methylation level according to (ii) and the Histone 3 trimethylation according to (iii).

More specifically, in the first step (a) of the method of the invention, the expression level of the biomarker of the invention, ARTS, is being determined. The terms “level of expression” or “expression level” are used interchangeably and generally refer to a numerical representation of the amount (quantity) of a polynucleotide which may be gene or an amino acid product or protein in a biological sample.

More specifically, “Expression” generally refers to the process by which gene-encoded information is converted into the structures present and operating in the cell. For example, gene expression values measured in Real-Time Polymerase Chain Reaction, sometimes also referred to as RT-PCR or quantitative PCR (qPCR), represent luminosity measured in a tested sample, where an intercalating fluorescent dye is integrated into double-stranded DNA products of the qPCR reaction performed on reverse-transcribed sample RNA, i.e., test sample RNA converted into DNA for the purpose of the assay. The luminosity is captured by a detector that converts the signal intensity into a numerical representation which is said expression value, in terms of gene. Therefore, according to the invention “expression” of a gene, specifically, a gene encoding ARTS may refer to transcription into a polynucleotide. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. Methods for determining the level of expression of the biomarkers of the invention will be described in more detail herein after.

In yet another embodiment, the first step of the method of the invention may require determining the methylation level of ARTS TSS. The term “methylation level” as used herein generally refers to a numerical representation of the amount (quantity) of a polynucleotide which is being methylated. In general, the term “methylation” refers to a process involving addition of methyl group. DNA methylation is one of several epigenetic mechanisms that cells use to control gene expression, specifically, in inhibiting gene expression. In the context of the invention, the methylation may involve addition of a methyl group to the DNA nucleotides cytosine or adenine. DNA methylation typically occurs in a CpG dinucleotide context.

The term “CpG islands” or CpG sites or CG sites as used herein denote “-C-phosphate-G-”, that is, cytosine and guanine separated by only one phosphate and refers to DNA regions wherein a cytosine nucleotide is present next to a guanine nucleotide in the linear sequence along the length. The “CpG” notation is used to distinguish linear sequence from the CG base pair of cytosine and guanine.

In some particular embodiments, the methylation level of the CpG islands at the Sept4/ARTS TSS is determined between positions +249 and +443 of Sept4/ARTS TSS. In more specific embodiments, the methylation may be determined in at least one position of +329, +339 and +406 of Sept4/ARTS TSS.

In yet another embodiment, the first step of the method of the invention requires determining the level of Histone H3 trimethylation. The term “level of histone trimethylation” as used herein generally refers to a numerical representation of the amount (quantity) of an amino acid which is be modified by the addition of one, two or three methyl groups. Specifically and in the context of the present application, the term “level of trimethylation” refers to a modification of a histone protein at different amino acids. For example, trimethylation of histone H3 may occurs at lysine 4 (H3K4), at lysine 9 (H3K9) or at lysine 27 (H3K27).

More specifically, the nucleosome, made up of four histone proteins (H2A, H2B, H3, and H4), is the primary building block of chromatin. Originally thought to function as a static scaffold for DNA packaging, histones have more recently been shown to be dynamic proteins, undergoing multiple types of post-translational modifications. One such modification, methylation of lysine residues, is a major determinant for formation of active and inactive regions of the genome. It should be noted that addition of methyl groups to histones by histone methyltransferases, can either activate or further repress transcription, depending on the amino acid being methylated and the presence of other methyl or acetyl groups in the vicinity.

In certain and specific embodiments, the method of the invention further comprises an additional and optional step of normalization.

According to this embodiment, in addition to determination the level of expression of ARTS and optionally of at least one of Survivin and AFP, the methylation of ARTS TSS or histone trimethylation, these examined parameters are also determined for at least one suitable control reference gene (actin for example) in the same sample.

More specifically, according to such embodiment, the expression level of ARTS and optionally of at least one of Survivin and AFP obtained in step (ai) may be normalized according to the expression level of said at least one reference control gene obtained in the additional optional step in said test sample, thereby obtaining a normalized expression value. Optionally, similar normalization is performed also in at least one control sample or a representing standard when applicable.

The term “expression value” refers to the result of a calculation, that uses as an input the “level of expression” or “expression level” obtained experimentally and by normalizing the “level of expression” or “expression level” by at least one normalization step as detailed herein, the calculated value termed herein “expression value” is obtained.

In yet another specific embodiment, in addition to determination Sept4/ARTS methylation level of the CpG islands at the TSS, the methylation level of at least one suitable control reference gene may be determined in the same sample. According to such embodiment, the methylation level at CpG islands at the TSS of Sept4/ARTS obtained in step (aii) may be normalized according to the methylation level of said at least one reference control gene obtained in the additional optional step in said test sample, thereby obtaining a normalized methylation value.

The term “value of Sept4/ARTS TSS methylation” refers to the result of a calculation, that uses as an input the “Sept4/ARTS methylation level” obtained experimentally and by normalizing the “Sept4/ARTS methylation level” by at least one normalization step as detailed herein, the calculated value termed herein “value of Sept4/ARTS TSS methylation” is obtained.

Still further, according to this embodiment, in addition to determination the level of Histone 3 trimethylation at at least one of lysine 4, lysine 9 and lysine 27, the level of Histone 3 trimethylation of at least one suitable control reference gene may be determined in the same sample. According to such embodiment, the level of Histone 3 trimethylation at at least one of lysine 4, lysine 9 and lysine 27 obtained in step (a iii) is normalized according to the level of trimethylation of said at least one reference control gene obtained in the additional optional step in said test sample, thereby obtaining a normalized trimethylation value.

The term “trimethylation value of histone H3” refers to the result of a calculation, that uses as an input the “the level of Histone 3 trimethylation” obtained experimentally and by normalizing the “the level of Histone 3 trimethylation” by at least one normalization step as detailed herein, the calculated value termed herein “trimethylation value of histone H3” is obtained.

It should be appreciated that an important step in the prognostic method of the inventions is determining whether the normalized expression value or value of methylation or trimethylation value of the biomarker of the invention, ARTS, is changed as compared to a pre determined cut off, or is within the range of such cutoff.

As noted above, the second step of the method of the invention involves calculating determining and comparing if the expression value obtained in step (a) is any one of, positive, negative or equal to a predetermined standard value (expression, methylation or histone trimethylation), or cutoff value. Such step involves calculating and measuring the difference between the expression, methylation or histone trimethylation values of the examined sample and the cutoff values and determining whether the examined sample can be defined as positive or negative.

As used herein the term “comparing” denotes any examination of the different parameters (expression, methylation or histone trimethylation) and/or values obtained in the samples of the invention as detailed throughout in order to discover similarities or differences between at least two different samples. It should be noted that comparing according to the present invention encompasses the possibility to use a computer based approach.

In yet more specific embodiments, the second step (b) of the method of the invention involves calculating and determining any one of:

(i) if the expression value of ARTS obtained in step (a i) is any one of, positive or negative with respect to a predetermined standard expression value of ARTS or to the expression value of ARTS in a control sample and optionally, determining if the expression value of at least one of Survivin and AFP is any one of, positive or negative with respect to a predetermined standard expression value of at least one of Survivin and AFP or to the expression value of at least one of Survivin and AFP in a control sample.
(ii) if the value of Sept4/ARTS TSS methylation obtained in step (a ii) is any one of, positive or negative with respect to a predetermined standard Sept4/ARTS TSS methylation or the Sept4/ARTS TSS methylation value in a control sample; and
(iii) if trimethylation value of histone H3 at said lysine residues obtained in step (a iii) is any one of, positive or negative with respect to a predetermined standard trimethylation value of histone H3 or to the trimethylation value in a control sample.

As described hereinabove, the method of the invention refers to predetermined values. It should be noted that a “predetermined value” is a value that meets the requirements for both high diagnostic sensitivity (true positive rate) and high diagnostic specificity (true negative rate).

It should be noted that the terms “sensitivity” and “specificity” are used herein with respect to the ability of the marker of the invention, to correctly classify a sample as belonging to a pre-established population associated with the existence of a disease or disorder or responsiveness to a specific treatment or to a specific relapse rate, as will be discussed herein after.

“Sensitivity” indicates the performance of the bio-marker of the invention, with respect to correctly classifying samples as belonging to pre-established populations that are likely to suffer from a disease or disorder or to respond to therapy or to relapse, when applicable, wherein said bio-marker are consider here as any of the options provided herein.

“Specificity” indicates the performance of the bio-marker of the invention with respect to correctly classifying samples as belonging to pre-established populations of subjects suffering from the same disorder or populations of subjects that are likely to respond to a specific treatment or unlikely to relapse as will be discussed herein after.

Simply put, “sensitivity” relates to the rate of correct identification of diagnosed, responsiveness and high-relapse rate samples as such out of a group of samples, whereas “specificity” relates to the rate of correct identification of lack of responsiveness and low-relapse rate samples as such out of a group of samples. Values may be used as a control sample, said values being the result of a statistical analysis of values in pre-established populations of healthy, responsive, nonresponsive, relapsed or remained disease-free (remission) subjects.

Thus, a given population having specific clinical parameters will have a defined likelihood to be diagnosed with a disease or disorder or respond or relapse based on the expression values of at least one of the markers being above or below said values.

For example, an individual having at least one of (i) a negative expression value of ARTS and optionally, a positive expression value of at least one of Survivin and AFP; (ii) a positive value of Sept4/ARTS TSS methylation; and (iii) a negative trimethylation value of histone H3 at lysine 4 or a positive value at lysine 9 or lysine 27; is considered to be suffering from a disease or a disorder, specifically a hepatic disorder and associated pathologies or of a solid proliferative disorder.

It should be emphasized that the nature of the invention is such that the accumulation of further patient data may improve the accuracy of the presently provided cutoff values.

The expression, methylation or histone trimethylation values are selected along ROC (Receiver Operating Characteristic) curve for optimal combination of prognostic sensitivity and prognostic specificity which are as close to 100 percent as possible, and the resulting values are used as the cutoff values that distinguish between healthy and diseased subjects, patients who will relapse at a certain rate, and those who will not (with said given sensitivity and specificity) and patients who will respond or not to a specific treatment. The ROC curve may evolve as more data of more patients and expression, methylation or histone trimethylation values are recorded and taken into consideration, modifying the optimal cutoff values and improving sensitivity and specificity. Thus, any cutoff values should be viewed as a starting point that may shift as more patient data allows more accurate cutoff value calculation.

As noted above, the expression, methylation or histone trimethylation values determined for the examined sample (or the normalized expression value) is compared with a predetermined cutoff or with the value obtained for a control sample. More specifically, in certain embodiments, the expression value obtained for the examined sample is compared with a predetermined standard or cutoff value.

In further embodiments, the predetermined standard value, or cutoff value has been pre-determined and calculated for a population comprising at least one of healthy subjects, subjects suffering from a hepatic or any solid proliferative disorder, subjects that respond to a specific treatment, non-responder subjects, subjects in remission and subjects in relapse.

Still further, in certain alternative embodiments where a control sample is being used (instead of, or in addition to, pre-determined cutoff values), the normalized expression values of the biomarker gene used by the invention in the test sample are compared to the values in the control sample. In certain embodiments, such control sample may be obtained from at least one of a healthy subject, a subject suffering from a disorder, a subject that responds to a specific treatment, a non-responder subject, a subject in remission and a subject in relapse.

“Standard” or a “predetermined standard” as used herein, denotes either a single standard value or a plurality of standards with which the level of ARTS expression, methylation and histone trimethylation from the tested sample is compared. The standards may be provided, for example, in the form of discrete numeric values or is calorimetric in the form of a chart with different colors or shadings for different levels of expression; or they may be provided in the form of a comparative curve prepared on the basis of such standards (standard curve).

As shown by the invention, ARTS inactivation occurs at pre malignant stages of HCC. HCC is an incurable disease that is usually detected at advanced stages, when surgical resection or liver transplantation, the most effective treatments for this malignancy, are no longer viable options. Although it is unknown at what precise point during tumorigenesis ARTS silencing occurs, the high rate of ARTS loss in HCC (FIG. 1) is consistent with the idea that ARTS inactivation occurs at a very early stage. Given that HCC is usually not diagnosed until advanced stages, monitoring ARTS expression may prove valuable as a clinical tool.

Thus, the present invention further provides the use of ARTS as an early marker for detecting early stages of a malignant process, or alternatively, early detection of relapse of patients treated with a certain therapeutic agent. In some embodiments, the method of the invention may be particularly suitable for monitoring and early diagnosis of relapse of the diagnosed disorder in the subject. More specifically, such method may further comprise several steps.

The third step (c), involves repeating step (a) of the diagnostic method of the invention described above, for at least one more temporally-separated test sample of the examined subject to obtain at least one of (i) the expression value of ARTS and optionally of at least one of Survivin and AFP; (ii) the value of Sept4/ARTS TSS methylation; and (iii) trimethylation value of histone H3 at said lysine residues, for at least one temporally separated sample.

In the next step (d), calculating the rate of change of at least one of (i) the expression value of ARTS and optionally of at least one of Survivin and AFP; (ii) the value of Sept4/ARTS TSS methylation; and (iii) trimethylation value of histone H3 at said lysine residues between said samples.

The further step (e) concerns determining if the rate of change calculated in step (d i-iii) is positive or negative with respect to a standard rate of change determined for a population of subjects suffering from said disorder in relapse and in remission or the rate of change obtained from at least one control sample.

It should be noted that wherein at least one of: (i) a negative rate of change of the expression value of ARTS and optionally, a positive rate of change of said expression value of at least one of Survivin and AFP; (ii) a positive rate of change in the value of Sept4/ARTS TSS methylation; and (iii) a negative rate of change in the trimethylation value of histone H3 at lysine 4 or a positive rate of change at lysine 9 or lysine 27; indicates that the examined subject is in relapse, thereby monitoring disease progression or providing an early prognosis for disease relapse.

Thus, according to such embodiments, the method of the invention further provides early prognosis/diagnosis for monitoring disease relapse.

The term “relapse”, as used herein, relates to the re-occurrence of a condition, disease or disorder that affected a person in the past. Specifically, the term relates to the re-occurrence of a disease being treated with a therapeutic agent.

“Prognosis” is defined as a forecast of the future course of a disease or disorder, based on medical knowledge. This highlights the major advantage of the invention, namely, the ability to predict relapse rate in patients as soon as they are diagnosed, even prior to treatment, based on a specific ARTS-related measured parameters (e.g., expression, methylation or histone trimethylation). This early prognosis facilitates the selection of appropriate treatment regimens that may minimize the predicted relapse, individually to each patient, as part of personalized medicine.

As indicated above, in accordance with some embodiments of the invention, in order to asses the patient condition, detect relapse or monitor the disease progression, as well as responsiveness to a certain treatment, at least two “temporally-separated” test samples must be collected from the examined patient and compared thereafter in order to obtain the rate of change in at least one of (i) the expression value of ARTS and optionally of at least one of Survivin and AFP; (ii) the value of Sept4/ARTS TSS methylation; and (iii) trimethylation value of histone H3 at said lysine residues between said samples. In practice, to detect a change in at least one of these parameters between said samples, at least two “temporally-separated” test samples and preferably more must be collected from the patient.

At least one of the following parameters, expression, methylation or histone trimethylation, is then determined using the method of the invention, applied for each sample. As detailed above, the rate of change in parameters is calculated by determining the ratio between at least two values of expression, methylation or histone trimethylation, obtained from the same patient in different time-points or time intervals.

This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least three days, at least one week, at least two weeks, at least three weeks, at least one month, at least two months, at least three months, at least four months, at least five months, at least one year, or even more.

In some embodiments, one of the time points may correspond to a period in which a patient is experiencing a remission of the disease.

The term “remission”, as used herein, relates to the state of absence of disease activity in patients known to have un-curable chronic illness. It is commonly used to refer to absence of active cancer or liver disorder when this disease is expected to manifest again in the future. A partial remission may be defined for cancer as 50 percent or greater reduction in the measurable parameters of tumor growth as may be found on physical examination, radiologic study, or by biomarker levels from a blood or urine test. A complete remission is defined as complete disappearance of all such manifestations of disease. Each disease or even clinical trial can have its own definition of a partial remission.

When calculating the rate of change, one may use any two samples collected at different time points from the patient. To ensure more reliable results and reduce statistical deviations to a minimum, averaging the calculated rates of several sample pairs is preferable. A calculated or average value of at least one of: (i) a negative rate of change of said expression value of ARTS and optionally, a positive rate of change of said expression value of at least one of Survivin and AFP; (ii) a positive rate of change in the value of Sept4/ARTS TSS methylation; and (iii) a negative rate of change in the trimethylation value of histone H3 at lysine 4 or a positive rate of change at lysine 9 or lysine 27; indicates that said subject is in relapse. It should be noted that in certain embodiments, where normalization step is being performed, the values referred to above, are normalized values.

As indicated above, in order to execute the prognostic method of the invention, at least two different samples must be obtained from the subject in order to calculate the rate of change as detailed above. By obtaining at least two and preferably more biological samples from a subject and analyzing them according to the method of the invention, the prognostic method may be effective for predicting, monitoring and early diagnosing molecular alterations indicating a relapse in said patient.

Thus, the prognostic method may be applicable for early, sub-symptomatic diagnosis of relapse when used for analysis of more than a single sample along the time-course of diagnosis, treatment and follow-up.

An “early diagnosis” provides diagnosis prior to appearance of clinical symptoms. Prior as used herein is meant days, weeks, months or even years before the appearance of such symptoms. More specifically, at least 1 week, at least 1 month, 2 months, 3 months, 4 months, 5 months, 6 months, 7 months, 8 months, 9 months, 10 months, 11 months, 12 months, or even few years before clinical symptoms appear.

The number of samples collected and used for evaluation of the subject may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more. Furthermore, to assess the trend in expression rates according to the invention, it is understood that the rate of change may be calculated as an average rate of change over at least three samples taken in different time points, or the rate may be calculated for every two samples collected at adjacent time points. It should be appreciated that the sample may be obtained from the monitored patient in the indicated time intervals for a period of several months or several years. More specifically, for a period of 1 year, for a period of 2 years, for a period of 3 years, for a period of 4 years, for a period of 5 years, for a period of 6 years, for a period of 7 years, for a period of 8 years, for a period of 9 years, for a period of 10 years, for a period of 11 years, for a period of 12 years, for a period of 13 years, for a period of 14 years, for a period of 15 years or more. In one particular example, the samples are taken from the monitored subject every two months for a period of 5 years.

The method for monitoring disease progression or early prognosis for disease relapse as detailed herein may be used for personalized medicine, by collecting at least two samples from the same patient at different stages of the disease.

As detailed herein, the method of the invention is also suitable for following the responsiveness of a patient to treatment at any time point after treatment. Accordingly, the patient may be evaluated in at least one time point after initiation of treatment in order to asses if the treatment protocol is efficient and appropriate. Determination can be carried out at an early time points such that a decision may be made regarding continuation of the treatment or alternatively readjusting the treatment protocol.

Thus, in yet other embodiments, the invention provides a method for assessing responsiveness of a mammalian subject to treatment with a specific therapeutic agent or evaluating the efficacy of treatment on a subject. This method is based on determining the expression, methylation or histone trimethylation values of the biomarkers of the invention before and after initiation of treatment, and calculating the ratio of the change in said values as a result of the treatment.

In more specific embodiments the method comprises the step of: First, in step (a), determining in a biological sample of the subject obtained prior to initiation of treatment at least one of:

(i) the level of expression of ARTS and optionally of at least one of Survivin and AFP to obtain an expression value.
(ii) Sept4/ARTS methylation level of the CpG islands at the TS S to obtain a value of Sept4/ARTS TS S methylation.
(iii) the level of Histone 3 trimethylation at at least one of lysine 4, lysine 9 and lysine 27 to obtain a trimethylation value of histone H3 at said lysine residues.

In the next step (b), repeating step (a) in at least one other biological sample of said subject obtained after initiation of said treatment.

The third step (c), involves calculating the rate of change of at least one of (i) the expression value of ARTS and optionally of at least one of Survivin and AFP; (ii) the value of Sept4/ARTS TS S methylation; and (iii) trimethylation value of histone H3 at said lysine residues between samples obtained before and after initiation of said treatment.

Finally, step (d) concerns determining if the rate of change calculated in step (c i-iii) is positive or negative with respect to a standard rate of change determined for a population of responder or and non-responder subjects suffering from the diagnosed disorder and treated with the examined therapeutic agent or the rate of change obtained from at least one control sample.

In certain embodiments it should be noted that wherein at least one of: (i) a negative rate of change of the expression value of ARTS and optionally, a positive rate of change of said expression value of at least one of Survivin and AFP; (ii) a positive rate of change in the value of Sept4/ARTS TSS methylation; and (iii) a negative rate of change in the trimethylation value of histone H3 at lysine 4 or a positive value at lysine 9 or lysine 27; indicates that the examined subject belongs to a pre-established population associated with lack of responsiveness to treatment with the therapeutic agent.

As used herein the term “assessing responsiveness” refers to determining the likelihood that the subject will respond to treatment, namely the success or failure of treatment.

The term “response” or “responsiveness” to treatment refers to an improvement in at least one relevant clinical parameter as compared to an untreated subject diagnosed with the same pathology (e.g., the same type, stage, degree and/or classification of the pathology), or as compared to the clinical parameters of the same subject prior to treatment.

The term “non responder” to treatment refers to a patient not experiencing an improvement in at least one of the clinical parameter and is diagnosed with the same condition as an untreated subject diagnosed with the same pathology (e.g., the same type, stage, degree and/or classification of the pathology), or experiencing the clinical parameters of the same subject prior to treatment.

As detailed above, the prediction obtained by the method of the invention made by comparing between the sample and the patient population may be dependent on the selection of population of patients to which the sample is compared to. A positive or higher expression value of the sample over a population of responders indicates that the examined subject is a responsive subject.

According to the present invention, the first step of the method of the invention involves determining in at least one biological sample of said subject at least one of:

(i) the level of expression of ARTS); (ii) Sept4/ARTS TSS methylation level; and (iii) the level of Histone 3 trimethylation. It should be noted that at least one sample should be obtained from said subject before the initiation of the treatment.

In some particular embodiments, determining the level of expression of ARTS and optionally of at least one of Survivin and AFP in a biological sample of the examined subject may be performed by the step of contacting detecting molecules specific for ARTS and optionally for at least one of Survivin and AFP with a biological sample of the subject, any aliquots thereof, or with any nucleic acid or protein product obtained therefrom.

In other embodiments, determining the level of Sept4/ARTS methylation level of the CpG islands at the TSS in a biological sample of said subject is performed by a methylation specific PCR of bisulfite-treated genomic DNA obtained from said sample. More specifically, such procedure may involve obtaining genomic DNA from at least one biological sample of said subject, treating the DNA with bisulfite, contacting said bisulfite treated DNA with methylation specific primers and performing methylation specific PCR.

In other embodiments, determining the level of Histone 3 trimethylation of at least one of lysine 4, lysine 9 and lysine 27 in a biological sample of said subject is performed by a chromatin immuno-precipitation assay of extracts of said sample. Non-limiting example for such procedure is provided in the examples section.

It should be noted that all optional steps for determining the different parameters indicated above, involve contacting the sample or any component thereof with a specific reagent (e.g., detecting molecules). The term “contacting” means to bring, put, incubates or mix together. As such, a first item is contacted with a second item when the two items are brought or put together, e.g., by touching them to each other or combining them. In the context of the present invention, the term “contacting” includes all measures or steps which allow interaction between the at least one of the detection molecules for ARTS, optionally for at least one of Survivin and AFP and optionally, for at least one suitable control reference gene and the nucleic acid or amino acid molecules of the tested sample. The contacting is performed in a manner so that the at least one of detecting molecule of for ARTS for example, can interact with or bind to the nucleic acid molecules or alternatively, a protein product of Sept4/ARTS gene, in the tested sample. The binding will preferably be non-covalent, reversible binding, e.g., binding via salt bridges, hydrogen bonds, hydrophobic interactions or a combination thereof.

In certain embodiments, the detection step further involves detecting a signal from the detecting molecules that correlates with the expression level of ARTS and optionally of at least one of Survivin and AFP in the sample from the subject, by a suitable means. According to some embodiments, the signal detected from the sample by any one of the experimental methods detailed herein below reflects the expression level of ARTS and optionally of at least one of Survivin and AFP. It should be noted that such signal-to-expression level data may be calculated and derived from a calibration curve.

Thus, in certain embodiments, the method of the invention may optionally further involve the use of a calibration curve created by detecting a signal for each one of increasing pre-determined concentrations of ARTS and optionally of at least one of Survivin and AFP. Obtaining such a calibration curve may be indicative to evaluate the range at which the expression levels correlate linearly with the concentrations of ARTS and optionally of at least one of Survivin and AFP. It should be noted in this connection that at times when no change in expression level of ARTS and optionally of at least one of Survivin and AFP is observed, the calibration curve should be evaluated in order to rule out the possibility that the measured expression level is not exhibiting a saturation type curve, namely a range at which increasing concentrations exhibit the same signal. It must be appreciated that in certain embodiments such calibration curve as described above may by also part or component in any of the kits provided by the invention as described herein after.

In other embodiments of the invention, the detecting molecules used for determining the expression levels ARTS according to the invention, may be either isolated detecting nucleic acid molecules or isolated detecting amino acid molecules. It should be noted that the invention further encompasses any combination of nucleic and amino acids for use as detecting molecules for the methods of the invention. As noted above, in the first step of the method of the invention, the sample or any nucleic acid or protein product derived therefrom is contacted with the detecting molecules of the invention.

In more specific embodiments, for determining the expression level of the biomarkers of the invention (e.g., ARTS and optionally, at least one of Survivin and AFP), nucleic acid detecting molecule may be used. More specifically, such nucleic acid detecting molecules may comprise isolated oligonucleotides, each oligonucleotide specifically hybridizes to a nucleic acid sequence of ARTS and optionally of at least one of Survivin and AFP. In an optional embodiment, where the expression levels of the biomarkers of the invention are normalized, the method of the invention may use nucleic acid detecting molecules specific for a control reference gene.

According to more specific embodiment, the nucleic acid detecting molecules used by the method of the invention may be at least one of a pair of primers or nucleotide probes.

As used herein, “nucleic acid molecules” or “nucleic acid sequence” are interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. A “nucleic acid” or “nucleic acid sequence” may also include regions of single- or double-stranded RNA or DNA or any combinations.

As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may be from about 3 to about 1,000 nucleotides long. Although oligonucleotides of 5 to 100 nucleotides are useful in the invention, preferred oligonucleotides range from about 5 to about 15 bases in length, from about 5 to about 20 bases in length, from about 5 to about 25 bases in length, from about 5 to about 30 bases in length, from about 5 to about 40 bases in length or from about 5 to about 50 bases in length. More specifically, the detecting oligonucleotides molecule used by the composition of the invention may comprise any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 bases in length. It should be further noted that the term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly.

As indicated throughout, in certain embodiments when the detecting molecules used are nucleic acid based molecules, specifically, oligonucleotides. It should be noted that the oligonucleotides used in here specifically hybridize to nucleic acid sequences of ARTS and optionally of at least one of Survivin and AFP. Optionally, where also the expression of at least one of ARTS and optionally of at least one of Survivin and AFP is being examined, the method of the invention may use as detecting molecules oligonucleotides that specifically hybridize to a nucleic acid sequence of said at least one for ARTS and optionally of at least one of Survivin and AFP genes. As used herein, the term “hybridize” refers to a process where two complementary nucleic acid strands anneal to each other under appropriately stringent conditions. Hybridizations are typically and preferably conducted with probe-length nucleic acid molecules, for example, 5-100 nucleotides in length, 5-50, 5-40, 5-30 or 5-20.

As used herein “selective or specific hybridization” in the context of this invention refers to a hybridization which occurs between a polynucleotide encompassed by the invention as detecting molecules, and ARTS and optionally of at least one of Survivin and AFP, wherein the hybridization is such that the polynucleotide binds to ARTS and optionally of at least one of Survivin and AFP or any control reference gene preferentially to any other RNA in the tested sample. In a specific embodiment a polynucleotide which “selectively hybridizes” is one which hybridizes with a selectivity of greater than 60 percent, greater than 70 percent, greater than 80 percent, greater than 90 percent and most preferably on 100 percent (i.e. cross hybridization with other RNA species preferably occurs at less than 40 percent, less than 30 percent, less than 20 percent, less than 10 percent). As would be understood to a person skilled in the art, a detecting polynucleotide which “selectively hybridizes” to ARTS and optionally of at least one of Survivin and AFP or any control reference gene can be designed taking into account the length and composition.

The terms, “specifically hybridizes”, “specific hybridization” refers to hybridization which occurs when two nucleic acid sequences are substantially complementary (at least about 60 percent complementary over a stretch of at least 5 to 25 nucleotides, preferably at least about 70 percent, 75 percent, 80 percent or 85 percent complementary, more preferably at least about 90 percent complementary, and most preferably, about 95 percent complementary).

The measuring of the expression of any one of ARTS and optionally of at least one of Survivin and AFP and any control reference gene can be done by using those polynucleotides as detecting molecules, which are specific and/or selective for ARTS and optionally of at least one of Survivin and AFP genes or any control reference gene to quantitate the expression of said ARTS and optionally of at least one of Survivin and AFP genes or any control reference gene. In a specific embodiment of the invention, the polynucleotides which are specific and/or selective for said ARTS and optionally of at least one of Survivin and AFP genes or any control reference gene may be probes or a pair of primers. It should be further appreciated that the methods, as well as the compositions and kits of the invention may comprise, as an oligonucleotide-based detection molecule, both primers and probes.

The term, “primer”, as used herein refers to an oligonucleotide, whether occurring naturally as in a purified restriction digest, or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 10-30 or more nucleotides, although it may contain fewer nucleotides. More specifically, the primer used by the methods, as well as the compositions and kits of the invention may comprise 10, 11, 12, 13, 14, 15, 16, 17, 18, 19, 20, 21, 22, 23, 24, 25, 26, 27, 28, 29 or 30 nucleotides or more. In certain embodiments, such primers may comprise 30, 40, 50, 60, 70, 80, 90, 100 nucleotides or more. In specific embodiments, the primers used by the method of the invention may have a stem and loop structure. The factors involved in determining the appropriate length of primer are known to one of ordinary skill in the art and information regarding them is readily available.

As used herein, the term “probe” means oligonucleotides and analogs thereof and refers to a range of chemical species that recognize polynucleotide target sequences through hydrogen bonding interactions with the nucleotide bases of the target sequences. The probe or the target sequences may be single- or double-stranded RNA or single- or double-stranded DNA or a combination of DNA and RNA bases. A probe may be 5, 6, 7, 8, 9, 10, 11, 12, 13, 14, 15, 16, 17, 18, 19, 20, 21, 22, 23, 24, 25, 26, 27, 28, 29 and up to 30 nucleotides in length as long as it is less than the full length of the target mRNA or any gene encoding said mRNA. Probes can include oligonucleotides modified so as to have a tag which is detectable by fluorescence, chemiluminescence and the like. The probe can also be modified so as to have both a detectable tag and a quencher molecule, for example TaqMan® and Molecular Beacon® probes, that will be described in detail below.

The oligonucleotides and analogs thereof may be RNA or DNA, or analogs of RNA or DNA, commonly referred to as antisense oligomers or antisense oligonucleotides. Such RNA or DNA analogs comprise, but are not limited to, 2-′0-alkyl sugar modifications, methylphosphonate, phosphorothiate, phosphorodithioate, formacetal, 3-thioformacetal, sulfone, sulfamate, and nitroxide backbone modifications, and analogs, for example, LNA analogs, wherein the base moieties have been modified. In addition, analogs of oligomers may be polymers in which the sugar moiety has been modified or replaced by another suitable moiety, resulting in polymers which include, but are not limited to, morpholino analogs and peptide nucleic acid (PNA) analogs. Probes may also be mixtures of any of the oligonucleotide analog types together or in combination with native DNA or RNA. At the same time, the oligonucleotides and analogs thereof may be used alone or in combination with one or more additional oligonucleotides or analogs thereof.

In some specific embodiments, primer-pairs for PCR may be any one of the following primers. In yet more specific embodiment, detecting molecules specific for ARTS may be oligonucleotides that specifically recognize and hybridize the ARTS nucleic acid sequence. Specific, particular and non limiting example for such detecting molecule may be denoted by SEQ ID NO. 1 and 2.

As a control, β-Actin primers may be used, for example, the primers of SEQ ID NO. 3 and SEQ ID NO. 4.

In certain embodiments, the use of ARTS as a biomarker is combined with AFP and/or Survivine. Accordingly, AFP primers may include SEQ ID NO. 7 and SEQ ID NO. 8 and in yet another embodiment, Survivin primers may include the primers of SEQ ID NO. 15 and SEQ ID NO. 16.

It should be appreciated that the detecting molecules described herein for ARTS are only non limiting examples. These examples may be also applicable for other aspects of the invention, namely, the compositions and kits described herein after.

As indicated above, using nucleic acid based detecting molecules, in some embodiments the level of expression of ARTS and optionally, of other biomarkers disclosed herein, may be determined using a nucleic acid amplification assay selected from the group consisting of: a Real-Time PCR, micro array, PCR, in situ hybridization and comparative genomic hybridization.

The term “amplification assay”, with respect to nucleic acid sequences, refers to methods that increase the representation of a population of nucleic acid sequences in a sample. Nucleic acid amplification methods, such as PCR, isothermal methods, rolling circle methods, etc., are well known to the skilled artisan. More specifically, as used herein, the term “amplified”, when applied to a nucleic acid sequence, refers to a process whereby one or more copies of a particular nucleic acid sequence is generated from a template nucleic acid, preferably by the method of polymerase chain reaction.

“Polymerase chain reaction” or “PCR” refers to an in vitro method for amplifying a specific nucleic acid template sequence. The PCR reaction involves a repetitive series of temperature cycles and is typically performed in a volume of 50-100 microliter. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers”, “a set of PCR primers” or “pair of primers” can comprise two, three, four or more primers.

Real time nucleic acid amplification and detection methods are efficient for sequence identification and quantification of a target since no pre-hybridization amplification is required. Amplification and hybridization are combined in a single step and can be performed in a fully automated, large-scale, closed-tube format.

Methods that use hybridization-triggered fluorescent probes for real time PCR are based either on a quench-release fluorescence of a probe digested by DNA Polymerase (e.g., methods using TaqMan®, MGB-TaqMan®), or on a hybridization-triggered fluorescence of intact probes (e.g., molecular beacons, and linear probes). In general, the probes are designed to hybridize to an internal region of a PCR product during annealing stage (also referred to as amplicon). For those methods utilizing TaqMan® and MGB-TaqMan® the 5′-exonuclease activity of the approaching DNA Polymerase cleaves a probe between a fluorophore and a quencher, releasing fluorescence.

Thus, a “real time PCR” or “RT-PCT” assay provides dynamic fluorescence detection of amplified ARTS or any control reference gene produced in a PCR amplification reaction. During PCR, the amplified products created using suitable primers hybridize to probe nucleic acids (TaqMan® probe, for example), which may be labeled according to some embodiments with both a reporter dye and a quencher dye. When these two dyes are in close proximity, i.e. both are present in an intact probe oligonucleotide, the fluorescence of the reporter dye is suppressed. However, a polymerase, such as AmpliTaq Gold™, having 5′-3′ nuclease activity can be provided in the PCR reaction. This enzyme cleaves the fluorogenic probe if it is bound specifically to the target nucleic acid sequences between the priming sites. The reporter dye and quencher dye are separated upon cleavage, permitting fluorescent detection of the reporter dye. Upon excitation by a laser provided, e.g., by a sequencing apparatus, the fluorescent signal produced by the reporter dye is detected and/or quantified. The increase in fluorescence is a direct consequence of amplification of target nucleic acids during PCR.

More particularly, QRT-PCR or “qPCR” (Quantitative RT-PCR), which is quantitative in nature, can also be performed to provide a quantitative measure of gene expression levels. In QRT-PCR reverse transcription and PCR can be performed in two steps, or reverse transcription combined with PCR can be performed. One of these techniques, for which there are commercially available kits such as TaqMan® (Perkin Elmer, Foster City, Calif.), is performed with a transcript-specific antisense probe. This probe is specific for the PCR product (e.g. a nucleic acid fragment derived from a gene) and is prepared with a quencher and fluorescent reporter probe attached to the 5′ end of the oligonucleotide. Different fluorescent markers are attached to different reporters, allowing for measurement of at least two products in one reaction.

When Taq DNA polymerase is activated, it cleaves off the fluorescent reporters of the probe bound to the template by virtue of its 5-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions can be performed in any solid support, for example, slides, microplates, 96 well plates, 384 well plates and the like so that samples derived from many individuals are processed and measured simultaneously. The TaqMan® system has the additional advantage of not requiring gel electrophoresis and allows for quantification when used with a standard curve.

A second technique useful for detecting PCR products quantitatively without is to use an intercalating dye such as the commercially available QuantiTect SYBR Green PCR (Qiagen, Valencia Calif.). RT-PCR is performed using SYBR green as a fluorescent label which is incorporated into the PCR product during the PCR stage and produces fluorescence proportional to the amount of PCR product.

Both TaqMan® and QuantiTect SYBR systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

Additionally, other known systems to quantitatively measure mRNA expression products include Molecular Beacons® which uses a probe having a fluorescent molecule and a quencher molecule, the probe capable of forming a hairpin structure such that when in the hairpin form, the fluorescence molecule is quenched, and when hybridized, the fluorescence increases giving a quantitative measurement of gene expression.

According to this embodiment, the detecting molecule may be in the form of probe corresponding and thereby hybridizing to any region or part of ARTS or any control reference gene. More particularly, it is important to choose regions which will permit hybridization to the target nucleic acids. Factors such as the Tm of the oligonucleotide, the percent GC content, the degree of secondary structure and the length of nucleic acid are important factors.

It should be further noted that a standard Northern blot assay can also be used to ascertain an RNA transcript size and the relative amounts of ARTS or any control gene product, in accordance with conventional Northern hybridization techniques known to those persons of ordinary skill in the art.

The invention further contemplates the use of amino acid based molecules such as proteins or polypeptides as detecting molecules disclosed herein and would be known by a person skilled in the art to measure the protein product of Sept4/ARTS gene. Techniques known to persons skilled in the art (for example, techniques such as Western Blotting, Immunoprecipitation, ELISAs, protein microarray analysis, Flow cytometry and the like) can then be used to measure the level of protein products corresponding to the biomarker of the invention. As would be understood to a person skilled in the art, the measure of the level of expression of the protein products of the biomarker of the invention, specifically, ARTS requires a protein, which specifically and/or selectively binds to the biomarker genes of the invention.

As indicated above, the detecting molecules of the invention may be amino acid based molecules that may be referred to as protein/s or polypeptide/s. As used herein, the terms “protein” and “polypeptide” are used interchangeably to refer to a chain of amino acids linked together by peptide bonds. In a specific embodiment, a protein is composed of less than 200, less than 175, less than 150, less than 125, less than 100, less than 50, less than 45, less than 40, less than 35, less than 30, less than 25, less than 20, less than 15, less than 10, or less than 5 amino acids linked together by peptide bonds. In another embodiment, a protein is composed of at least 200, at least 250, at least 300, at least 350, at least 400, at least 450, at least 500 or more amino acids linked together by peptide bonds. It should be noted that peptide bond as described herein is a covalent amid bond formed between two amino acid residues.

In specific embodiments, the detecting amino acid molecules are isolated antibodies, with specific binding selectively to the ARTS protein. Using these antibodies, the level of expression of ARTS protein may be determined using an immunoassay which is selected from the group consisting of FACS, a Western blot, an ELISA, a RIA, a slot blot, a dot blot, immunohistochemical assay and a radio-imaging assay.

The term “antibody” as used in this invention includes whole antibody molecules as well as functional fragments thereof, such as Fab, F(ab)2, and Fv that are capable of binding with antigenic portions of the target polypeptide, i.e. ARTS protein. The antibody is preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” or “monoclonal antibody composition”, which as used herein refer to a preparation of antibodies or fragments thereof of single molecular composition.

It should be recognized that the antibody can be a human antibody, a chimeric antibody, a recombinant antibody, a humanized antibody, a monoclonal antibody, or a polyclonal antibody.

The antibody can be an intact immuno globulin, e.g., an IgA, IgG, IgE, IgD, IgM or subtypes thereof. The antibody can be conjugated to a functional moiety (e.g., a compound which has a biological or chemical function.

As noted above, the term “antibody” also encompasses antigen-binding fragments of an antibody. The term “antigen-binding fragment” of an antibody (or simply “antibody portion,” or “fragment”), as used herein, may be defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab′)2 is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”, or ScFv), a genetically engineered molecule containing the variable region of the light chain and the variable region of the heavy chain, linked by a suitable polypeptide linker as a genetically fused single chain molecule.

Methods of generating such antibody fragments are well known in the art (See for example, Harlow and Lane, Antibodies: A Laboratory Manual, Cold Spring Harbor Laboratory, New York, 1988, incorporated herein by reference).

Purification of serum immunoglobulin antibodies (polyclonal antisera) or reactive portions thereof can be accomplished by a variety of methods known to those of skill in the art including, precipitation by ammonium sulfate or sodium sulfate followed by dialysis against saline, ion exchange chromatography, affinity or immuno-affinity chromatography as well as gel filtration, zone electrophoresis, etc.

Still further, for diagnostic and monitoring uses described herein after, the anti-ARTS proteins antibodies used by the present invention may optionally be covalently or non-covalently linked to a detectable label. The term “labeled” can refer to direct labeling of the antibody via, e.g., coupling (i.e., physically linking) a detectable substance to the antibody, and can also refer to indirect labeling of the antibody by reactivity with another reagent that is directly labeled. Examples of indirect labeling include detection of a primary antibody using a fluorescently labeled secondary antibody. More specifically, detectable labels suitable for such use include any composition detectable by spectroscopic, photochemical, biochemical, immunochemical, electrical, optical or chemical means. Useful labels in the present invention include magnetic beads (e.g. DYNABEADS), fluorescent dyes (e.g., fluorescein isothiocyanate, Texas red, rhodamine, green fluorescent protein, and the like), radiolabels (e.g., ³H, ¹²⁵I, ³⁵S, ¹⁴C, or ³²P), enzymes (e.g., horseradish peroxidase, alkaline phosphatase and others commonly used in an ELISA and competitive ELISA and other similar methods known in the art) and colorimetric labels such as colloidal gold or colored glass or plastic (e.g. polystyrene, polypropylene, latex, etc.) beads.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The antibody used as a detecting molecule according to the invention, specifically recognizes and binds ARTS protein. It should be therefore noted that the term “binding specificity”, “specifically binds to an antigen”, “specifically immuno-reactive with”, “specifically directed against” or “specifically recognizes”, when referring to an epitope, specifically, a recognized epitope within the ARTS protein, refers to a binding reaction which is determinative of the presence of the epitope in a heterogeneous population of proteins and other biologics. More particularly, “selectively bind” in the context of proteins encompassed by the invention refers to the specific interaction of a any two of a peptide, a protein, a polypeptide an antibody, wherein the interaction preferentially occurs as between any two of a peptide, protein, polypeptide and antibody preferentially as compared with any other peptide, protein, polypeptide and antibody.

Thus, under designated immunoassay conditions, the specified antibodies bind to a particular epitope at least two times the background and more typically more than 10 to 100 times background. More specifically, “Selective binding”, as the term is used herein, means that a molecule binds its specific binding partner with at least 2-fold greater affinity, and preferably at least 10-fold, 20-fold, 50-fold, 100-fold or higher affinity than it binds a non-specific molecule.

A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

According to one embodiment, where amino acid-based detection molecules are used, the expression level of the ARTS protein, in the tested sample can be determined using different methods known in the art, specifically method disclosed herein below as non-limiting examples.

Enzyme-Linked Immunosorbent Assay (ELISA) is used herein involves fixation of a sample containing a protein substrate (e.g., fixed cells or a proteinaceous solution) to a surface such as a well of a microtiter plate. A substrate-specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western Blot as used herein involves separation of a substrate from other protein by means of an acryl amide gel followed by transfer of the substrate to a membrane (e.g., nitrocellulose, nylon, or PVDF). Presence of the substrate is then detected by antibodies specific to the substrate, which are in turn detected by antibody-binding reagents. Antibody-binding reagents may be, for example, protein A or secondary antibodies. Antibody-binding reagents may be radio labeled or enzyme-linked, as described hereinafter. Detection may be by autoradiography, colorimetric reaction, or chemiluminescence. This method allows both quantization of an amount of substrate and determination of its identity by a relative position on the membrane indicative of the protein's migration distance in the acryl amide gel during electrophoresis, resulting from the size and other characteristics of the protein.

In one version, Radioimmunoassay (RIA) involves precipitation of the desired protein (i.e., the substrate) with a specific antibody and radio labeled antibody-binding protein (e.g., protein A labeled with I¹²⁵) immobilized on a perceptible carrier such as agars beads. The radio-signal detected in the precipitated pellet is proportional to the amount of substrate bound.

In an alternate version of RIA, a labeled substrate and an unlabelled antibody-binding protein are employed. A sample containing an unknown amount of substrate is added in varying amounts. The number of radio counts from the labeled substrate-bound precipitated pellet is proportional to the amount of substrate in the added sample.

Fluorescence-Activated Cell Sorting (FACS) involves detection of a substrate in situ in cells bound by substrate-specific, fluorescently labeled antibodies. The substrate-specific antibodies are linked to fluorophore. Detection is by means of a flow cytometry machine, which reads the wavelength of light emitted from each cell as it passes through a light beam. This method may employ two or more antibodies simultaneously, and is a reliable and reproducible procedure used by the present invention.

Immunohistochemical Analysis involves detection of a substrate in situ in fixed cells by substrate-specific antibodies. The substrate specific antibodies may be enzyme-linked or linked to fluorophore. Detection is by microscopy, and is either subjective or by automatic evaluation. With enzyme-linked antibodies, a calorimetric reaction may be required. It will be appreciated that immunohistochemistry is often followed by counterstaining of the cell nuclei, using, for example, Hematoxyline or Giemsa stain.

It should be appreciated that all the detecting molecules used by any of the methods, as well as the compositions and kits of the invention described herein after, are isolated and/or purified molecules. As used herein, “isolated” or “purified” when used in reference to a nucleic acid means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95% pure) of non-nucleotide material naturally associated with it, and thus is distinguished from isolated chromosomes. As used herein, the terms “isolated” and “purified” in the context of a proteinaceous agent (e.g., a peptide, polypeptide, protein or antibody) refer to a proteinaceous agent which is substantially free of cellular material and in some embodiments, substantially free of heterologous proteinaceous agents (i.e. contaminating proteins) from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous proteinaceous agent (e.g. protein, polypeptide, peptide, or antibody; also referred to as a “contaminating protein”). When the proteinaceous agent is recombinantly produced, it is also preferably substantially free of culture medium, i.e. culture medium represents less than about 20%, 10%, or 5% of the volume of the protein preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. Preferably, proteinaceous agents disclosed herein are isolated.

Still further, according to certain embodiments, the methods of the invention may use any appropriate biological sample. The term “biological sample” in the present specification and claims is meant to include samples obtained from a mammalian subject.

According to an embodiment of the invention, the sample may be a blood sample which can be obtained using a syringe needle from a vein of the subject. It should be noted that the cell may be isolated from the subject (e.g., for in vitro detection) or may optionally comprise a cell that has not been physically removed from the subject (e.g., in vivo detection).

According to a specific embodiment, the sample used by the method of the invention may be a sample of peripheral blood mononuclear cells (PBMCs).

The phrase, “peripheral blood mononuclear cells (PBMCs)” as used herein, refers to a mixture of monocytes and lymphocytes. Several methods for isolating white blood cells are known in the art. For example, PBMCs can be isolated from whole blood samples using density gradient centrifugation procedures. Typically, anticoagulated whole blood is layered over the separating medium. At the end of the centrifugation step, the following layers are visually observed from top to bottom: plasma/platelets, PBMCs, separating medium and erythrocytes/granulocytes. The PBMC layer is then removed and washed to remove contaminants (e.g., red blood cells) prior to determining the expression level of the polynucleotide(s) bio-markers of the invention.

In yet another embodiment, the sample may be a biopsy of human organs or tissue, specifically, liver biopsy. The biopsies may be obtained by a surgical operation from an organ or tissue of interest, for example liver biopsy, breast biopsy, etc.

The term biopsy used herein refers to a medical test commonly performed by a surgeon or an interventional radiologist involving sampling of cells or tissues for examination. It is the medical removal of tissue from a living subject to determine the presence or extent of a disease. The tissue is generally examined under a microscope by a pathologist, and can also be analyzed chemically. When an entire lump or suspicious area is removed, the procedure is called an excisional biopsy. When only a sample of tissue is removed with preservation of the histological architecture of the tissue's cells, the procedure is called an incisional biopsy or core biopsy. When a sample of tissue or fluid is removed with a needle in such a way that cells are removed without preserving the histological architecture of the tissue cells, the procedure is called a needle aspiration biopsy.

It should be noted that although organ biopsies and blood samples are presently used herein, certain embodiments of the invention contemplate the use of any biological samples. The term “sample” in the present specification and claims is meant to include biological samples. Biological samples may be obtained from mammal, specifically, a human subject, include fluid, solid (e.g., stool) or tissues. The term “sample” may also include body fluids such as whole blood sample, blood cells, bone marrow, lymph fluid, serum, plasma, urine, sputum, saliva, faeces, semen, spinal fluid or CSF, the external secretions of the skin, respiratory, intestinal, and genitourinary tracts, tears, milk, any human organ or tissue, any biopsy, for example, lymph node or spleen biopsies, any sample taken from any tissue or tissue extract, any sample obtained by lavage optionally of the breast ductal system, plural effusion, samples of in vitro or ex vivo cell culture and cell culture constituents. Some samples that are a priori not liquid are contacted with a liquid buffers which are then used according to the diagnostic method of the invention.

As detailed herein and shown in Example 1, the level of ARTS is reduced in patients suffering from HCC. Thus, the methods of the invention may be particularly applicable for diagnosis, and as disclosed herein after, treatment of hepatic disorders as well as of solid tumors.

In some embodiments, the hepatic disorder and associated pathologies that may be diagnosed and treated by any of the methods provided by the invention may be any one of hepatocellular carcinoma (HCC), hepatitis, Non-alcoholic steatohepatitis (NASH), fatty liver disease, hepatic steatosis, the metabolic syndrome, steatohepatitis, liver cirrhosis and liver fibrosis.

Hepatocellular carcinoma (HCC) also known as malignant hepatoma is the most common type of liver cancer. HCC is often secondary to either a viral hepatitis infection (hepatitis B or C) or cirrhosis (alcoholism being the most common cause of hepatic cirrhosis). Primary liver cancer most commonly manifests as hepatocellular carcinoma and/or cholangiocarcinoma; rarer forms include angiosarcoma and hemangiosarcoma of the liver. In certain embodiments, the methods and kits of the invention are applicable for primary HCC. It should be noted that many liver malignancies are secondary lesions that have metastasized from primary cancers in the gastrointestinal tract and other organs, such as the kidneys, lungs, breast, or prostate. Therefore, the method of the invention may further be applicable for secondary hepatic malignancies.

In yet another embodiment, the diagnostic, prognostic and therapeutic methods of the invention may be applicable for hepatitis. Hepatitis (plural hepatitides) is a medical condition defined by the inflammation of the liver and characterized by the presence of inflammatory cells in the tissue of the organ. Hepatitisis caused mainly by various viruses (viral hepatitis) but also by some liver toxins (e.g. alcoholic hepatitis), autoimmunity (autoimmune hepatitis) or hereditary conditions.

In yet another embodiment, the diagnostic, prognostic and therapeutic methods of the invention may be applicable for liver cirrhosis and liver fibrosis. Cirrhosis as used herein, is the formation of fibrous tissue (fibrosis) in the place of liver cells that have died due to a variety of causes, including viral hepatitis, alcohol overconsumption, and other forms of liver toxicity. Cirrhosis causes chronic liver failure.

In yet another embodiment, the diagnostic, prognostic and therapeutic methods of the invention may be applicable for Fatty liver disease. Fatty liver disease (hepatic steatosis) is a reversible condition where large vacuoles of triglyceride fat accumulate in liver cells. Non-alcoholic fatty liver disease is a spectrum of disease associated with obesity and metabolic syndrome, among other causes. Fatty liver may lead to inflammatory disease (i.e. steatohepatitis) and, eventually, cirrhosis.

In yet another embodiment, the diagnostic, prognostic and therapeutic methods of the invention may be applicable for the Metabolic Syndrome or any of the conditions comprising the same, for example, at least one of dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability, blood fibrinolysis defects and hypertension.

Still further, as a hepatic disorder, the methods provided by the invention may be also applicable for hepatic disorders caused by a viral infection, for example, HCV infection. The phrase “HCV infection” encompasses acute (refers to the first 6 months after infection) and chronic (refers to infection with hepatitis C virus which persists more than 6 month) infection with the hepatitis C virus. Thus, according to some embodiments of the invention, the subject is diagnosed with chronic HCV infection.

According to some embodiments of the invention, the subject is infected with HCV type 1. According to some embodiments of the invention, the subject is infected with HCV type 2, 3 or 4.

Still further, in some embodiments, the methods of the invention may be used for the diagnosis of solid proliferative disorders. Solid tumors refer to a solid mass of cancer cells that grow in organ systems and can occur anywhere in the body. Two types of solid tumors are seen in adults: epithelial tumors and sarcomas. Epithelial tumors, which can also be called carcinomas, account for 90% of the solid tumors people have, and they occur in the lining (epithelium) that is on the outside or inside of the organ. For instance, the digestive system, which starts in the mouth and ends at the anus, is lined all the way down with an epithelium. Sarcomas are also called “connective tissue cancer” because they occur in the tissue that keeps the organs together. Connective tissues are the muscles, tendons, fat, nerves and other tissues that connect, support or surround structures and organs in the body. Sarcomas are usually named for the type of tissue where they first occur. For instance, bone tumors are called osteosarcomas.

It should be noted that a proliferative disorder as used herein, encompasses malignant and non-malignant solid proliferative disorders.

As used herein, “proliferative disorder” is a disorder displaying hyper proliferation. This term means cell division and growth that is not part of normal cellular turnover, metabolism, growth, or propagation of the whole organism. Unwanted proliferation of cells is seen in tumors and other pathological proliferation of cells, does not serve normal function, and for the most part will continue unbridled at a growth rate exceeding that of cells of a normal tissue in the absence of outside intervention. A pathological state that ensues because of the unwanted proliferation of cells is referred herein as a “hyper proliferative disease” or “hyper proliferative disorder.” It should be noted that the term “proliferative disorder”, “cancer”, “tumor” and “malignancy” all relate equivalently to a hyperplasia of a tissue or organ.

As used herein to describe the present invention, “cancer”, “tumor” and “malignancy” all relate equivalently to a hyperplasia of a tissue or organ. Malignancies of tissues or organs may produce solid tumors. If the tissue is a part of the lymphatic or immune systems, malignant cells may include non-solid tumors of circulating cells. In general, the diagnostic, prognostic and therapeutic methods of the present invention may be applicable for patients suffering of solid tumors.

In some embodiments, the solid proliferative disorder may be any one of carcinoma, melanoma, sarcoma, glioma and blastoma.

Carcinoma as used herein, refer to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Further malignancies that may find utility in the present invention can comprise but are not limited to solid tumors (including GI tract, colon, lung, liver, breast, prostate, pancreas and Kaposi's sarcoma). More particularly, the malignant disorder that may be diagnosed and treated according to the invention include solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

In yet another aspect, the invention provides a method for treating, preventing, ameliorating or delaying the onset of a hepatic disorder and associated pathologies or of a solid proliferative disorder in a subject in need thereof. In certain embodiments, the method of the invention may comprise the steps of:

In the first step (a), determining in at least one biological sample of the subject at least one of:

(i) the level of expression of Apoptosis Related Protein in the TGF-beta Signaling Pathway (ARTS) and optionally of at least one of Survivin and α-fetoprotein (AFP) to obtain an expression value;
(ii) Sept4/ARTS methylation level of the CpG islands at the TS S to obtain a value of Sept4/ARTS TS S methylation; and
(iii) the level of Histone 3 trimethylation at at least one of lysine 4, lysine 9 and lysine 27 to obtain a trimethylation value of histone H3 at said lysine residues;

In the second step (b), determining at least one of:

(i) if the expression value of ARTS obtained in step (a i) is any one of, positive or negative with respect to a predetermined standard expression value of ARTS or the expression value of ARTS in a control sample and optionally, determining if the expression value of at least one of Survivin and AFP is any one of, positive or negative with respect to a predetermined standard expression value of at least one of Survivin and AFP or to the expression value of at least one of Survivin and AFP in a control sample;
(ii) if the value of Sept4/ARTS TSS methylation obtained in step (a ii) is any one of, positive or negative with respect to a predetermined standard Sept4/ARTS TSS methylation or to the Sept4/ARTS TSS methylation value in a control sample;
(iii) if trimethylation value of histone H3 at said lysine residues obtained in step (a iii) is any one of, positive or negative with respect to a predetermined standard trimethylation value of histone H3 or to the trimethylation value in a control sample.

The third step (c) involves administering a therapeutically effective amount of at least one chromatin modifying drug or ARTS or any fragment, peptide, analogues and derivatives thereof or any composition comprising the same, to a subject displaying at least one of (i) a negative expression value of ARTS and optionally, a positive expression value of at least one of Survivin and AFP; (ii) a positive value of Sept4/ARTS TSS methylation; and (iii) a negative trimethylation value of histone H3 at lysine 4 or a positive value at lysine 9 or lysine 27 as determined in step (b).

In certain embodiments, the at least one chromatin-modifying drug may be a drug that inhibits DNA methylation. In more specific embodiments, a chromatin-modifying drug useful in the invention may be any one of 5′ aza-cytosine (AZA) and trichostatin A (TSA).

As mentioned above, the method of the invention is specifically intended for treating any hepatic disorder and associated pathologies. In non-limiting examples, such hepatic disorders may be any one of HCC, hepatitis, NASH, fatty liver disease, hepatic steatosis, the metabolic syndrome, steatohepatitis, liver cirrhosis and liver fibrosis.

In yet another embodiment, the method of the invention may be suitable for treating a solid proliferative disorder, for example, any one of carcinoma, melanoma, sarcoma, glioma and blastoma. In should be noted that such solid proliferative disorder is characterized by loss of ARTS expression.

As noted above, the therapeutic method of the invention involves a preliminary diagnostic step that in some embodiments, involves determining the methylation level of the CpG islands at the Sept4/ARTS TSS is determined between positions +249 and +443. In more particular embodiments, the methylation may be determined in at least one position of +329, +339 and +406 of Sept4/ARTS TSS.

Still further, in some embodiments, the diagnostic step of the therapeutic method of the invention may involve determining the level of expression of ARTS and optionally of at least one of Survivin and AFP in a biological sample of the subject. In more specific embodiments, such determination may be performed by the step of contacting detecting molecules specific for ARTS and optionally for at least one of Survivin and AFP with a biological sample of the subject, or with any nucleic acid or protein product obtained therefrom.

In yet another particular embodiment, where the diagnostic step of the therapeutic method of the invention involves determining the level of Sept4/ARTS methylation, the level of the methylation of CpG islands at ARTS TSS in a biological sample of the subject is performed by a methylation specific PCR of bisulfite-treated genomic DNA obtained from said sample.

In other embodiments, where the diagnostic step of the therapeutic method of the invention involves determining the level of Histone 3 trimethylation of at least one of lysine 4, lysine 9 and lysine 27 in a biological sample of said subject, such determination may be performed by a chromatin immuno-precipitation assay of extracts of said sample.

It should be appreciated that the diagnostic step of the method of treatment provided by the invention is performed in a biological sample that may be any one of a tissue biopsy and a blood sample.

As indicated above, the invention described herein encompasses methods for the treatment of subjects in need thereof. The term “treatment” concerns improvement of at least one undesired manifestation of the disease such as: increase in disease free periods, decrease in acute disease periods (in time and severely), decrease in severity of the disease, improvement in life quality, decreased mortality, decrease in the rate of disease progression as well as prophylactic treatment before disease occurs.

More specifically, the term “treatment or prevention” as used herein, refers to the complete range of therapeutically positive effects of administrating to a subject including inhibition, reduction of, alleviation of, and relief from, a hepatic disorder or any related condition and illness, symptoms or undesired side effects or related disorders. More specifically, treatment or prevention of relapse re recurrence of the disease in response to a treatment with a non-effective, or deleterious therapeutic agent, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

By “patient” or “subject in need” it is meant any mammal who may be affected by the above-mentioned conditions, and to whom the treatment and diagnosis methods herein described is desired, including human, bovine, equine, canine, murine and feline subjects. Preferably said patient is a human. Administering of the therapeutic agent to the patient includes both self-administration and administration to the patient by another person. It should be noted that treatment according to the invention, would ameliorate or decrease in acute disease periods (in time and severely), decrease in severity of the disease, or even prevent.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. In some embodiments this term refers to an amount of a compound or composition which is administered to a subject in need thereof, necessary to effect a beneficial change in the severity of a disease or disorder, or prevent such disease, in said subject. This amount should also be within specific pharmacological ranges, to avoid toxic effects by over-dosing.

As indicated above, the invention described herein encompasses methods for the treatment of subjects in need thereof. The term “treatment” concerns improvement of at least one undesired manifestation of the disease such as: increase in disease free periods, decrease in acute disease periods (in time and severely), decrease in severity of the disease, improvement in life quality, decreased mortality, decrease in the rate of disease progression as well as prophylactic treatment before disease occurs.

In yet a further aspect, the invention provides a kit comprising means for performing at least two of:

(a) determining the level of expression of ARTS and optionally of at least one of Survivin and AFP in a biological sample;
(b) determining the level of Sept4/ARTS methylation of the CpG islands at the TSS in a biological sample; and
(c) determining the level of Histone 3 trimethylation of at least one of lysine 4, lysine 9 and lysine 27 in a biological sample.

According to one embodiment, means for determining the level of expression of ARTS and optionally of at least one of Survivin and AFP may comprise:

(a) detecting molecules specific for ARTS;
(b) optionally, detecting molecules specific for at least one of Survivin and AFP;
(c) predetermined calibration curve providing standard expression values of ARTS;
(d) optionally, predetermined calibration curve providing standard expression values of at least one of Survivin and AFP.

In certain embodiments, such detecting molecules may be nucleic acid based molecules or amino-acid based molecules. In more specific embodiments, nucleic acid detecting molecule comprises isolated oligonucleotides (primers or probes), each oligonucleotide specifically hybridizes to a nucleic acid sequence of ARTS and optionally, to a control reference gene. In yet another embodiment, an amino-acid based molecule may be an antibody specific for ARTS.

It should be noted that in certain embodiments, the kit of the invention may comprise, in case of nucleic acid detecting molecules, pairs of primers and optionally, also probes specific for the markers of the invention (ARTS and optionally, Survivine and AFP), as well as for control molecule (having a similar expression level in malignant and healthy tissue). It should be noted that the kit of the invention may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 250, 300, 350, 400, 450, 500 or more pairs of primers and optionally, probes. Still further, the kit of the invention may comprise up to 500 pairs of primers and optionally, probes. In yet another embodiment, the kit of the invention may comprise more than 1, 2, 3, 4, 5, 6, 7, 8, 9, 10 or more different pairs of primers and optionally, probes specific for ARTS or specific for the other markers used by the invention. In other specific embodiments, the kit of the invention may comprise up to 100 different pairs of primers and optionally, probes specific for ARTS, optionally for the other markers used by the invention and also specific for controls, such as beta-actin.

In certain embodiments the kit of the invention may comprise means for determining the level of Sept4/ARTS methylation of the CpG islands at the TSS may comprise:

(a) methylation specific primers;
(b) optionally, reagents for extracting genomic DNA from a sample;
(c) bisulfite containing reagent; and
(d) predetermined calibration curve providing standard values of Sept4/ARTS TSS methylation.

In yet another specific embodiment, the kit of the invention may comprise means for determining the level of Histone 3 trimethylation of at least one of lysine 4, lysine 9 and lysine 27. In more specific embodiments such means may comprise:

(a) at least one of anti-H3K4me3, anti-H3K27me3 and anti-H3K9me3 antibodies;
(b) primers for amplifying genomic intervals of Sept4/ARTS; and
(c) predetermined calibration curve providing standard values of Histone 3 trimethylation of at least one of lysine 4, lysine 9 and lysine 27.

In some embodiments, the kit of the invention may be particularly suitable for use in a method for the diagnosis and prognosis of hepatic disorder and associated pathologies or of a solid proliferative disorder in a mammalian subject.

In yet another embodiment, the kit of the invention may be used in monitoring and early diagnosis of relapse of a hepatic disorder and associated pathologies or of a solid proliferative disorder in said subject.

Still further, in some embodiments the kit of the invention may be for use in a method for determining the efficacy and assessing responsivness of a mammalian subject suffering from a hepatic disorder and associated pathologies or of a solid proliferative disorder to treatment with a therapeutic agent.

The invention further provides a kit suitable for therapeutic purposes. In such embodiments, the kit may further comprise at least one of: a therapeutically effective amount of at least one chromatin modifying drug; and ARTS or any fragment, peptide, analogues and derivatives thereof or any composition comprising the same.

In more specific embodiments such kit may be particularly useful in a method for treating, preventing, ameliorating or delaying the onset of a hepatic disorder and associated pathologies or of a solid proliferative disorder in a subject in need thereof.

In more specific embodiments such at least one chromatin-modifying drug may be a drug that inhibits DNA methylation. In more specific embodiments the chromatin-modifying drug may be any one of AZA and TSA.

In yet another embodiment, the kit of the invention may be adapted for examining different biological samples. In some embodiments the biological sample may be any biopsy of any human organ or tissue, a whole blood sample or any blood cells.

According to specific embodiments, the biological sample may be a blood sample. The kit of the invention may therefore optionally comprise suitable mans for obtaining said sample. More specifically, for using the kit of the invention, one must first obtain samples from the tested subjects. To do so, means for obtaining such samples may be required. Such means for obtaining a sample from the mammalian subject (a) can be any means for obtaining a sample from the subject known in the art. Examples for obtaining e.g. blood samples are known in the art and could be any kind of finger or skin prick or lancet based device, which basically pierces the skin and results in a drop of blood being released from the skin. In addition, aspirating or biopsy needles may be also used for obtaining spleen lymph nodes tissue samples. Samples may of course be taken from any other living tissue, or body secretions comprising viable cells, such as biopsies, saliva or even urine.

The inventors consider the kit of the invention in compartmental form. It should be therefore noted that the detecting molecules or any reagents used for determining expression, methylation or histone trimethylation in connection with ARTS, may be provided in a kit attached to an array. As defined herein, a “detecting molecule array” refers to a plurality of detection molecules that may be nucleic acids based (primers or probes), or protein based detecting molecules (specifically, antibodies), optionally attached to a support where each of the detecting molecules is attached to a support in a unique pre-selected and defined region.

For example, an array may contain different detecting molecules, such as specific antibodies or primers. It should be noted that each detecting molecule may be spatially arranged in a predetermined and separated location in an array. For example, an array may be a plurality of vessels (test tubes), plates, micro-wells in a micro-plate, each containing different detecting molecules or reagents. An array may also be any solid support holding in distinct regions (dots, lines, columns) different and known, predetermined detecting molecules.

It should be appreciated that in certain embodiments, the invention further provides an array comprising a plurality of detecting molecules for the markers of the invention, specifically, ARTS, and optionally for survivine and/or AFP, as well as for control reference markers.

As used herein, “solid support” is defined as any surface to which molecules may be attached through either covalent or non-covalent bonds. Thus, useful solid supports include solid and semi-solid matrixes, such as aerogels and hydrogels, resins, beads, biochips (including thin film coated biochips), microfluidic chip, a silicon chip, multi-well plates (also referred to as microtiter plates or microplates), membranes, filters, conducting and nonconducting metals, glass (including microscope slides) and magnetic supports. More specific examples of useful solid supports include silica gels, polymeric membranes, particles, derivatized plastic films, glass beads, cotton, plastic beads, alumina gels, polysaccharides such as Sepharose, nylon, latex bead, magnetic bead, paramagnetic bead, superparamagnetic bead, starch and the like. This also includes, but is not limited to, microsphere particles such as Lumavidin™ or LS-beads, magnetic beads, charged paper, Langmuir-Bodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is also contemplated. This includes surfaces with any topology, including, but not limited to, spherical surfaces and grooved surfaces.

It should be further appreciated that any of the reagents, substances or ingredients included in any of the methods and kits of the invention may be provided as reagents embedded, linked, connected, attached, placed or fused to any of the solid support materials described above.

All scientific and technical terms used herein have meanings commonly used in the art unless otherwise specified. The definitions provided herein are to facilitate understanding of certain terms used frequently herein and are not meant to limit the scope of the present disclosure.

As used herein the term “about” refers to ±10% The terms “comprises”, “comprising”, “includes”, “including”, “having” and their conjugates mean “including but not limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

### RT-PCR Analysis of Liver Cancer Biopsies and Cells

Human cancer panels containing arrayed cDNAs, derived from pathology-verified biopsies and non-tumor controls, were purchased from Origene (Rockville, Md.). qPCR was performed using SYBR Green incorporation. Primer composition and additional details for all PCR reactions is provided herein:

Quantitative reverse transcription polymerase chain reactions (qRT-PCR) incorporating SYBR Green (Applied Biosystems) were carried-out using a Realplex qPCR Mastercycler (Eppendorf; Hauppauge, N.Y.). For semi-quantitative PCR, total RNA was extracted from cell-lines using Trizol reagent (Invitrogen, Carlsbad, Calif.) and in turn, was used to synthesize cDNA using random primers, reagents, and protocol provided in the Superscript 1^{st} strand synthesis kit (Invitrogen). Normalized amounts of cDNA served as template in standard PCR using AmpliTaq DNA polymerase (Applied Biosystems).

### Primer-Pairs for PCR are the Following:

ARTS (Sept4_i2)
SEQ ID NO. 1
5′-GAGACGAGAGTGGCCTGAACCGA-3′
and
SEQ ID NO. 2
5′-AACAGGAACCTGTGACCACCTGC-3′,;
β-Actin
(5′-CATCCTGCGTCTGGACCTGGCTGG-3′ SEQ ID NO. 3
and
5′-CTAGAAGCATTTGCGGTGGACGAT-3′) SEQ ID NO. 4;
H5 (Sept4_il):
(5′-GAGGACAGGACTGAAGCTGGGAT-3′ SEQ ID NO. 5
and
5′-GGTTGCAAAGCCCACATACTCCT-3′) SEQ ID NO. 6,
AFP
(5′-TGAAAACCCTCTTGAATGCCAAG-3′ SEQ ID NO. 7
and
5′-GCCACAGGCCAATAGTTTGTCCT-3′) SEQ ID NO. 8:
Omi/Htr2A
(5′-ACATCCGGCATTGTTAGCTC-3′ SEQ ID NO. 9
and
5′TTCTTCTTTTCCCCACGATG-3′) SEQ ID NO. 10;
Smac/Diablo
(5′-GGAAAGCAGAAACCAAGCTG-3′ SEQ ID NO. 11
and
5′-AAAATGCTTTGGGTGTGAGG-3′) SEQ ID NO. 12;
Siah1
(5′-TGACTATGTGTTACCGCCCA-3′ SEQ ID NO. 13
and
5′-ATCAGATGGGGCATTACAGC-3′) SEQ ID NO. 14;
Survivin
(5′-CCACTGAGAACGAGCCAGACTT-3′ SEQ ID NO. 15
and
5′-GACAGAAAGGAAAGCGCAACCG-3′) SEQ ID NO. 16.

### Animals and Cell-Lines

Chemical induction of HCC was achieved via intraperitoneal injection of DEN (Sigma) in 2-week old male mice, at a dosage of 25 mg/kg body weight, and was followed by PB supplementation in drinking water (0.7%).

Septin-4 deficient mice were generated by homologous recombination, as previously detailed (26), and backcrossed eight generations into the C57BL/6J strain before use in the experiments described herein. Albumin-Myc (Alb-Myc) transgenic mice were originally generated by the lab of Dr. S. Thorgeirsson at the NIH. Animals were maintained at the Comparative Bioscience Center at RU and all mouse experiments were conducted in accordance with institutional and NIH guidelines. Hepatocyte cell-lines FL-62891, THLE-2, THLE-3, SK-Hep1, and PLC/PRF/5 were obtained from the American Tissue Culture Collection (ATCC; Rockville, Md.). PH5CH cells were kindly provided by Dr. M. Noguchi (Univ. of Tsukuba, Japan). HepG2, Hep3B, and Huh7 were gifts from Dr. Charles Rice's lab at RU. All cell-lines were cultivated in 10% fetal bovine serum with antibiotics (lx penicillin/streptomycin). FL-62891, PH5CH, Huh7, and THLE-2/-3 were cultured in Isocove's medium (ATCC), DMEM:Ham's F12 (1:1), MEM Eagle's (ATCC), or HCM supplemented with bulletkit (Lonza; Allendale, N.J.), respectively. The previously generated p53^{-/-}-immortalized PHM-1 murine hepatoblasts, expressing Myc-IRES-GFP (30), were maintained in DMEM. All other cell-lines were cultured in media recommended by the ATCC.

Chemically-induced and oncogene-driven HCC and analysis of liver cancer Chemical induction of HCC was achieved with a single intraperitoneal injection of DEN (Sigma) in 2-week old male offspring of Sept4^{+/-}×Sept4^{+/-} matings, at a dosage of 25 mg/kg body weight, and was followed by PB supplementation in drinking water at a concentration of 0.7% starting at 1-month of age. At the indicated ages (9, 10, or 12 months), mice were sacrificed and their liver weighed, lesion(s) counted and measured to calculate volume. Livers were subsequently formalin-fixed, paraffin-embedded, sectioned, and hematoxylin & eosin stained for histological diagnosis by certified pathologists (S.S.C. and L.J.). For oncogene-driven HCC, Alb-Myc male mice were crossed with Sept4^{-/-} females, producing F1-progeny Alb-Myc, Sept^{+/-} animals. The livers of 12- to 15-month old male Alb-Myc, Sept^{+/+} and Alb-Myc, Sept4^{-/-} mice in the F2 generation, which were offspring derived from F1-sibling matings, were analyzed as described above.

### Genomic DNA Bisulfite Conversion and Methylation Analysis

Genomic DNA from cell-lines, isolated using the DNeasy kit from Qiagen (Valencia, Calif.), was bisulfite-converted with the EZ DNA Methylation-Gold Kit, according to manufacturer's instructions.

In turn, bisulfite-treated DNA served as template for methylation-specific (MS)-PCR or direct sequencing analyses. For the former, the following methylation-(M) and unmethylated-(U) specific primer pairs were used:

(M) forward
SEQ ID NO. 17
5′-AGTTATGGTAAGGTAGGTAGGTAGGC-3
and
reverse
SEQ ID NO. 18
5′-AAAAAAAACTTAAAAACAACGACG-3′;
(U)forward
SEQ ID NO. 19
5′-TATGGTAAGGTAGGTAGGTAGGTGG-3′
and
reverse
SEQ ID NO. 20
5′-AAAAAAACTTAAAAACAACAACACA-3′.

For direct-sequencing, amplicons were PCR-generated, gel-excised, and ligated to TOPO-plasmid (Invitrogen).

Primers for PCR amplification are the following:

forward
SEQ ID NO. 21
5′-GGGTTGTAGTTATGGTAAGGTAGGTAG-3′
and
reverse
SEQ ID NO. 22
5′-ACCCAAAATAAATAAATAAAAAAAA-3′.

In turn, sequence information of individually cloned alleles was obtained through automated sequencing performed by Genewiz (South Plainfield, N.J.), following bacterial transformation and plasmid recovery using QIAprep (Qiagen).

### Short Hairpin RNA, Retroviral Transduction, Hepatoblast Sub-Cutaneous Transplantation, and Cell Death Assay

miR30-designed shRNAs were subcloned into the pLMP (MSCV-based) retroviral vector as previously described (30). Briefly, individual shRNAs were synthesized as 97 base-pair oligos (Sigma Genosys), PCR-amplified, ligated into pLMP following EcoRI/XhoI-digestion.

The oligos for generation of specific shRNA's are the following:

pan-Sept4
SEQ ID NO. 23
TGCTGTTGACAGTGAGCGCAAGCACGCAGTGGATATAGAATAGTGAAGC
CACAGATGTATTCTATATCCACTGCGTGCTTATGCCTACTGCCTCGGA;
H5-1
SEQ ID NO. 24
TGCTGTTGACAGTGAGCGCCAGGACCAAGCCCTAAAGGAATAGTGAAGC
CACAGATGTATTCCTTTAGGGCTTGGTCCTGTTGCCTACTGCCTCGGA;
H5-2
SEQ ID NO. 25
TGCTGTTGACAGTGAGCGAAAGGAACGGAATCGCAACAAATAGTGAAGC
CACAGATGTATTTGTTGCGATTCCGTTCCTTCTGCCTACTGCCTCGGA;
H5-3
SEQ ID NO.26
TGCTGTTGACAGTGAGCGCCAGATGAAGGAGACTCACTAATAGTGAAGC
CACAGATGTATTAGTGAGTCTCCTTCATCTGTTGCCTACTGCCTCGGA;
ARTS-1
SEQ ID NO. 27
TGCTGTTGACAGTGAGCGCAAGGGAACAGGCCGAGTAGCATAGTGAAGC
CACAGATGTATGCTACTCGGCCTGTTCCCTTATGCCTACTGCCTCGGA;
ARTS-2
SEQ ID NO. 28
TGCTGTTGACAGTGAGCGAAACAGGCCGAGTAGCACCACATAGTGAAGC
CACAGATGTATGTGGTGCTACTCGGCCTGTTCTGCCTACTGCCTCGGA;
ARTS-3
SEQ ID NO. 29
TGCTGTTGACAGTGAGCGACGGGTGGTCACTGATTCCTGTTAGTGAAGC
CACAGATGTAACAGGAATCAGTGACCACCCGCTGCCTACTGCCTCGGA.

Details on isolation, culture, retrovirus-mediated gene transfer, and sub-cutaneous transplantation of hepatoblasts can be found in (30). Cell viability was monitored by propidium iodide (PI) nuclear staining and flow cytometric analysis.

### Plasmids and Site-Directed Mutatgenesis

To generate MSCV-ARTS-c/Flag wob (Hygro), the murine arts cDNA was first PCR-amplified from the FirstChoice PCR-Ready Mouse Liver cDNA library (Ambion; Austin, Tex.), digested with KpnI and XbaI, and ligated into the corresponding sites in pcDNA3.1-c/Flag (Pham et al. (2004) Cell 119: 529), creating an in-frame, C-terminally Flag-epitope tagged ARTS (pcDNA3.1-ARTSc/Flag). Primers used for PCR-amplification are the following:

sense
SEQ ID NO. 30
5′-GGAGGTACCACCATGGATGACAAGGAGTATGTGGGCTTT-3′
and
anti-sense
SEQ ID NO. 31
5′-GGATCTAGAACATCCAATGGCCGGAGCCTGGGGAACAG-3′.

The cDNA was sequenced and verified to match the mouse ARTS coding sequence (GenBank CF553913.1). Next, wobble mutations were introduced into the coding sequence in this plasmid using the QuickChange site-directed mutagenesis kit (Stratagene, La Jolla, Calif.), according to manufacturer's instructions, and the following primers 5′-GGCAGGGCTACCACAAGTGGATGGTCGCTCATTCCTGTTCCCCAG-3′ SEQ ID NO. 32 and 5′-CTGGGGAACAGGAATGAGCGACCATCCACTTGTGGTAGCCCTGCC-3′ SEQ ID NO. 33. In turn, this plasmid was digested with PmeI, and the resulting 0.6 kilo base-pair fragment was gel-isolated, and ligated to HpaI-restriction digested MSCV (Hygro), generating MSCV-ARTS-c/Flag wob (Hygro), as confirmed by sequencing and restriction digest analyses.

### Chromatin Immunoprecipitation (ChIP), Immunoblotting, and Reagents

Native ChIP was performed as previously detailed in (27). qPCR analysis incorporating SYBR Green was performed using StepOnePlus Real-Time PCR System (Applied Biosystems). Primer sets amplifying the genomic interval of Sept4/ARTS with respect to the transcriptional start site, shown in parenthesis below, are the following:

A (-893 to -822)
forward
SEQ ID NO. 34
5′-CCTGACCAGCTCAGACCCTACT-3′
and
reverse
SEQ ID NO. 35
5′-GAAGGCAGATGCTTGGTGAGT-3′;
B (-820 to -759)
forward
SEQ ID NO. 36
5′-CCTAGCTGCTGCTCTGACCTTT-3′
and
reverse
SEQ ID NO. 37
5′-GACAGATGCCCCAGAAGCTTT-3′;
C (-651 to -589)
forward
SEQ ID NO. 38
5′-AAGGCTACACCGCCCTGTTA-3′
and
reverse
SEQ ID NO. 39
5′-GGATCATCTGCTGGAAGGATAGA-3′;
D (-602 to -534)
forward
SEQ ID NO. 40
5′-CCAGCAGATGATCCCTGGAT-3′
and
reverse
SEQ ID NO. 41
5′-TCATGCAGTACCCCAGATTACAGA-3′;
E (-457 to -394)
forward
SEQ ID NO. 42
5′-GTAGTGATCAGTAACTTGGGCATCA-3′
and
reverse
SEQ ID NO. 43
5′-CGCAGGGATTCAACAAGGTT-3′;
F (-302 to -236)
forward
SEQ ID NO. 44
5′-TTCTTTGGGAACTGGCTCAAG-3′
and
reverse
SEQ ID NO. 45
5′-TGGACACCTGGCTCTTTTTTACT-3′);
G (+446 to +529)
forward
SEQ ID NO. 46
5′-CTGGGTCAGCTTTTACCTGAGTAAC-3′
and
reverse
SEQ ID NO. 47
5′-GCGTTTTCCAAATGAAGTTGGT-3′);
H (+708 to +842)
forward
SEQ ID NO. 48
5′-ACAGGAACCATGGGAAAGCTT-3′
and
reverse
SEQ ID NO. 49
5′-GGGTTACTGTTTCTCTTCCCACTACA-3′;
I (+862 to +927)
forward
SEQ ID NO. 50
5′-TGCCCAGGTAGGAAGTTTTTGA-3′
and
reverse
SEQ ID NO. 51
5′-GGCAACAATGTCAGGAGAAGAGA-3′;
J (+997 to +1055)
forward
SEQ ID NO. 52
5′-GGAAGCCCCACAGCCTTT-3′
and
reverse
SEQ ID NO. 53
5′-CCCCCTGCTGTAAACAGTTTG-3′.

Immunoblotting was performed as previously described in (7). Cell extracts were prepared from tumor and adjacent non-tumor control tissues that were excised from the liver of sacrificed mice, homogenized by Polytron PT 2100 (Brinkman) in Triton X-100 lysis buffer and clarified by centrifugation. Extracts were normalized for protein concentration by Bradford assay (BioRad) prior to immunoblotting procedures. Signal detection was performed with enhanced chemiluminescence kit (Amersham). The following primary antibodies were used: anti-A6 (gift from Dr. V. Factor NCI/NIH), anti-Flag and anti-β-actin (Sigma); anti-XIAP (Becton Dickinson); anti-cIAP-1 (R&D Systems); anti-β-catenin and anti-Survivin (Cell Signaling), anti-cytokeratin-8 (Fitzgerald Indust. Intl., Concord, Mass.). 5-Aza-2′deoxycytidine (AZA), Trichostatin A (TSA), and cycloheximide (CHX) were purchased from Sigma-Alrich. Tumor-necrosis factor-α, the anti-Fas antibody (Jo-2), and the in vitro methylated genomic Hela DNA were obtained from Peprotech, Pharminogen, and New England Biolabs, respectively.

Antibodies for ChIP assays are the following: anti-H3K4me3 (Active Motif, 39159), anti-H3K27me3 (Abcam, 6002), and anti-H3K9me3 (Active Motif, 39161).

### Example 1

### Sept4/ARTS but not Sept4/H5 is Frequently Lost in Human HCC

Expression of ARTS is not restricted to the hematopoietic system and ARTS is highly expressed in a broad range of tissues (26). To examine the role of ARTS in solid tumors such as HCC, ARTS mRNA expression was determined in biopsies derived from liver cancer patients (FIG. 1A and Table 1). Whereas 10% of such samples displayed near-normal levels and another 10% had increased ARTS, 80% showed a significant reduction (FIG. 1A). Among the biopsies displaying reduced ARTS, most expressed less than one-third the normal levels, with many showing only trace amounts. Thus, expression of ARTS is significantly reduced in the majority of HCC patients.

TABLE 1
ARTS expression is frequently lost in patient HCC tumor material

| | | | | | | | Relative ARTS | |
|---|---|---|---|---|---|---|---|---|
| | | | Ct-Value | Ct-Value | ΔCt | ΔΔCt | (Fold vs Norm.) | |
| Sample No. | Sex | Age (yr.) | (ARTS) | (β-Actin) | (ARTS-β-Actin | (Cancer-Ref.) | 2^{-ΔΔCt}) | Diagnosis |
| Norm. 1^{a} | M | 81 | 33.85 | 24.9 | 8.95 | | | N.A. |
| Norm. 2^{b} | M | 73 | 33.05 | 24.24 | 8.81 | | | N.A. |
| Norm. 3^{c} | M | 71 | 33.38 | 24.83 | 8.55 | | | N.A. |
| Norm. 4^{d} | M | 86 | 33.92 | 24.69 | 9.23 | | | N.A. |
| Norm. 5 | M | 52 | 35.04 | 24.5 | 10.54 | | | N.A. |
| Norm. 6 | F | 33 | 33.39 | 24.58 | 8.81 | | | N.A. |
| Norm. 7^{e} | M | 66 | 34.72 | 24.44 | 10.28 | | | N.A. |
| Norm. 8^{f} | M | 68 | 33.12 | 24.81 | 8.31 | | | N.A. |
| Avg. | | | 33.81 ± 0.73 | 24.62 ± 0.23 | 9.19 ± 0.81 | | | |
| Patient 1^{a} | M | 81 | 33.81 | 24.77 | 9.04 | −0.15 | 1.110 | HCC |
| Patient 2 | F | 79 | 34.1 | 24.88 | 9.22 | 0.03 | 0.979 | HCC |
| Patient 3 | F | 58 | 34.15 | 24.92 | 9.23 | 0.04 | 0.973 | HCC |
| Patient 4 | M | 79 | 34.09 | 24.79 | 9.3 | 0.11 | 0.927 | HCC |
| Patient 5 | M | 56 | 14.99 | 24.59 | −9.6 | −18.79 | 453266 | HCC |
| Patient 6^{b} | M | 73 | 29.89 | 24.53 | 5.36 | −3.83 | 14.221 | HCC |
| Patient 7^{c} | M | 86 | 32.28 | 24.42 | 7.86 | −1.33 | 2.514 | HCC |
| Patient 8 | M | 26 | 33.43 | 24.75 | 8.68 | −0.51 | 1.424 | HCC |
| Patient 9 | M | 68 | 33.99 | 24.63 | 9.36 | 0.17 | 0.889 | HCC |
| Patient 10 | M | 50 | 34.56 | 25.04 | 9.52 | 0.33 | 0.796 | HCC |
| Patient 11 | M | 60 | 33.74 | 24.12 | 9.62 | 0.43 | 0.742 | HCC |
| Patient 12 | F | 58 | 35.21 | 25.29 | 9.92 | 0.73 | 0.603 | HCC |
| Patient 13 | M | 77 | 34.69 | 24.64 | 10.05 | 0.86 | 0.551 | HCC |
| Patient 14 | F | 31 | 35.41 | 25.33 | 10.08 | 0.89 | 0.540 | Adenoma |
| Patient 15 | M | 63 | 35.19 | 24.98 | 10.21 | 1.02 | 0.493 | HCC |
| Patient 16 | M | 71 | 35.64 | 25.41 | 10.23 | 1.04 | 0.486 | HCC |
| Patient 17 | M | 43 | 35.4 | 25.15 | 10.25 | 1.06 | 0.480 | HCC |
| Patient 18 | M | 70 | 34.89 | 24.37 | 10.52 | 1.33 | 0.398 | HCC |
| Patient 19 | M | 73 | 35.34 | 24.78 | 10.56 | 1.37 | 0.387 | HCC |
| Patient 20 | M | 79 | 35.1 | 24.47 | 10.63 | 1.44 | 0.369 | HCC |
| Patient 21^{g} | M | 66 | 35.6 | 24.76 | 10.84 | 1.65 | 0.319 | HCC |
| Patient 22^{f} | M | 68 | 35.27 | 24.38 | 10.89 | 1.70 | 0.308 | HCC |
| Patient 23 | M | 43 | 35.75 | 24.75 | 11 | 1.81 | 0.285 | HCC |
| Patient 24 | F | 39 | 36.09 | 24.92 | 11.17 | 1.98 | 0.253 | HCC |
| Patient 25^{c} | M | 71 | 36.03 | 24.78 | 11.25 | 2.06 | 0.240 | HCC |
| Patient 26 | F | 32 | 36.28 | 24.8 | 11.48 | 2.29 | 0.204 | Hyperplasia |
| Patient 27 | M | 26 | 35.88 | 24.37 | 11.51 | 2.32 | 0.200 | HCC |
| Patient 28 | M | 77 | 36.76 | 25.15 | 11.61 | 2.42 | 0.187 | HCC |
| Patient 29^{e} | M | 66 | 36.44 | 24.72 | 11.72 | 2.53 | 0.173 | HCC |
| Patient 30 | M | 68 | 36.95 | 25.06 | 11.89 | 2.70 | 0.154 | HCC |
| Patient 31 | M | 56 | 36.15 | 24.16 | 11.99 | 2.80 | 0.144 | HCC |
| Patient 32 | F | 63 | 36.51 | 24.43 | 12.08 | 2.89 | 0.135 | HCC |
| Patient 33 | M | 21 | 36.42 | 24.26 | 12.16 | 2.97 | 0.128 | HCC |
| Patient 34 | F | 61 | 37.66 | 25.48 | 12.18 | 2.99 | 0.126 | HCC |
| Patient 35 | F | 79 | 36.99 | 24.58 | 12.41 | 3.22 | 0.107 | HCC |
| Patient 36 | M | 60 | 36.51 | 24 | 12.51 | 3.32 | 0.100 | HCC |
| Patient 37 | F | 62 | 37.29 | 24.78 | 12.51 | 3.32 | 0.100 | HCC |
| Patient 38 | M | 60 | 37.63 | 24.88 | 12.75 | 3.56 | 0.085 | HCC |
| Patient 39 | F | 62 | 36.71 | 23.86 | 12.85 | 3.66 | 0.079 | CCC |
| Patient 40 | F | 78 | 40.2 | 24.54 | 15.66 | 6.47 | 0.011 | CCC |

Abbreviations: male (M), female (F), not applicable (N.A.), not reported (N.R.), hepatocellular carcinoma (HCC), and cholangiocarcinoma (CCC).

Table 1 shows results of quantitative reverse-transcription PCT (qRT-PCR) measuring ARTS and β-actin mRNA in biopsies from liver cancer-patients and non-tumor control tissues. Cycle threshold (Ct) values are reported for each bio-specimen. For calculation of relative normalized ARTS expression (Fold vs. Normal), the 2^{-ΔΔct} method was used: the average ΔCt value of normal tissues served as reference (Ref.). Matched tumor and adjacent non-tumor biopsies corresponding to the same individual are indicated (^{a-f}). The biopsy from Patent 21 was exercised from a lung mass and determined to be metastatic of liver origin (^{g}).

Next, the frequency of patients showing reduced ARTS was compared with those displaying upregulation of Survivin and α-fetoprotein (AFP), two widely accepted biomarkers of HCC (25, 28). Within the group of 40 biopsies from above, 27 (68%) and 18 (45%) specimens displayed a 2-fold or higher than normal expression of Survivin and AFP mRNA, respectively (FIGS. 1B and C). Nearly all of the samples showed a loss of ARTS or an increase in Survivin or AFP (FIGS. 1A-C: all but patients 3, 7, and 8), and 12 (30%) had abnormal levels of all three mRNAs. Of note, nearly one-fifth of the biopsies showed no upregulation of Survivin nor AFP, yet had reduced ARTS mRNA (FIGS. 1A-C; patient nos. 11, 14, 16, 20, 26, 30, and 31). Hence, in cases where Survivin and AFP fail to coincide with malignancy, ARTS mRNA levels still provided an indication of liver cancer. Thus, ARTS may be a useful biomarker for the diagnosis of liver cancer, in particular in combination with AFP and Survivin.

The expression of different Sept4-isoforms was next examined in hepatocyte cell-lines. Levels of ARTS mRNA in HCC cell-lines were dramatically reduced in comparison to non-malignant hepatocyte cell-lines and reached a 5- to 10-fold reduction (FIGS. 1D and 1E). Again, ARTS loss correlated with up-regulation of AFP (FIG. 1F); although in SK-Hep1 cells, little AFP could be detected. In contrast to ARTS, mRNA levels of Sept4/H5, Smac/Diablo, Omi/Htr2A, and Siah1 were not different between control and HCC cell-lines (FIG. 1F). Interestingly, only very low levels of ARTS were detected in FL62891, a fetal liver cell-line (FIGS. 1D and 1E). Together, these results indicate that ARTS loss is a common occurrence in HCC and could be associated with progression of the disease.

### Example 2

### The Sept4/ARTS but not the Sept4/H5 Promoter is Inactivated by DNA Hypermethylation and Site-Specific Histone Trimethylation in HCC

To understand how ARTS is lost in HCC, the DNA sequence of the Sept4/ARTS promoter (FIG. 2A) was examined. As shown in FIG. 2B, a high density of CpG dinucleotides, putative targets of DNA methylation (29), forms a predicted CpG island surrounding the ARTS-specific transcriptional start site (TSS). In contrast, the promoter of Sept4/H5 appears devoid of any CpG island (FIG. 2E). These observations suggest that epigenetic mis-regulation may account for ARTS loss in HCC.

To examine the possibility of epigenetic regulation, chromatin-modifying drugs were tested for their ability to influence ARTS expression. Cells were exposed to DNA-methyltransferase and HDAC inhibitors 5′ aza-cytosine (AZA) and trichostatin A (TSA), respectively. In HCC cell-lines, individual and co-treatments of AZA and TSA induced ARTS mRNA to levels comparable to those observed in normal hepatocytes (FIG. 2C). In contrast, AZA and TSA had little appreciable effect on ARTS levels in PH5CH (FIG. 2C). Thus, ARTS silencing in tumor cells is reversible and may be explained largely by DNA hypermethylation and chromatin remodeling.

To determine if ARTS repression involves modification of the Sept4/ARTS promoter, bisulfite-conversion studies were performed. First, methylation-specific PCR (MS-PCR) indicated that DNA hypermethylation occurs directly in the CpG-rich region of the Sept4/ARTS TSS (FIGS. 2B and 2D). Subsequently, direct sequencing analysis identified the most frequently methylated CpGs reside between positions +249 and +443, with CpGs at +329, +339 and +406 methylated most often, at frequencies ranging between 80 to 100% (FIGS. 3A-3D). In contrast, considerably less methylation was detected here in PH5CH and THLE-3, and little, if any, in THLE-2 (FIGS. 3B and 3C), thus, coinciding with high ARTS levels in these cell-lines (FIGS. 1D and 1E). Therefore, in HCC cells, ARTS-specific promoter hypermethylation contributes to the reduction of ARTS expression.

However, DNA methylation alone was insufficient to explain all the observations herein. In particular, SK-Hep1 cells showed only a modest amount of DNA methylation (FIGS. 3B and 3D), suggesting that other mechanisms may contribute to decreasing ARTS. To investigate this further, trimethylation of histone-3 (H3) at lysines-4, -9, and -27 (henceforth, H3K4me3, H3K9me3, and H3K27me3) was next examined. Whereas H3K4me3 is closely associated with transcriptional competence and activation, with peak levels usually appearing near the TSS in highly active genes (31, 32), H3K27me3 and H3K9me3 are most often linked with distinct types of gene silencing (31, 33).

Chromatin immunopreprecipitation assays revealed that H3K4me3 levels accumulate near the TSS of Sept4/ARTS (FIG. 3E). In HCC cell-lines, H3K4me3 levels here were decreased by half or more in comparison to normal hepatocytes (FIG. 3E). Furthermore, in agreement with their known function as more repressive chromatin signatures, the patterns of H3K27me3 and H3K9me3 were increased in HCC cell-lines and most prominently in SK-Hep1 cells, where they were elevated to approximately 10-fold higher than normal (FIGS. 3F and 3G). Thus, in SK-Hep1, despite the near normal amounts of DNA methylation and H3K4me3 (FIGS. 3B, 3D, and 3E), high levels of H3K27me3 and H3K9me3 (FIGS. 3F and 3G, respectively) in the Sept4/ARTS promoter were likely to play an important role in ARTS down-regulation. It may be concluded that inactivation of the Sept4/ARTS promoter can involve a combination of DNA hypermethylation and site-selective histone methylation.

### Example 3

### Sept4-Null Mice Show Increased Susceptibility to HCC

The functional significance of ARTS inactivation to cancer formation was next examined. To this end, Sept4-null and control littermate mice were monitored for emergence of HCC after being subjected to exposure to tumor initiating and promoting agents, diethylnitrosamine (DEN) and phenobarbital (PB), respectively. This widely used chemically-induced HCC rodent model mirrors an aggressive form of the human disease (34).

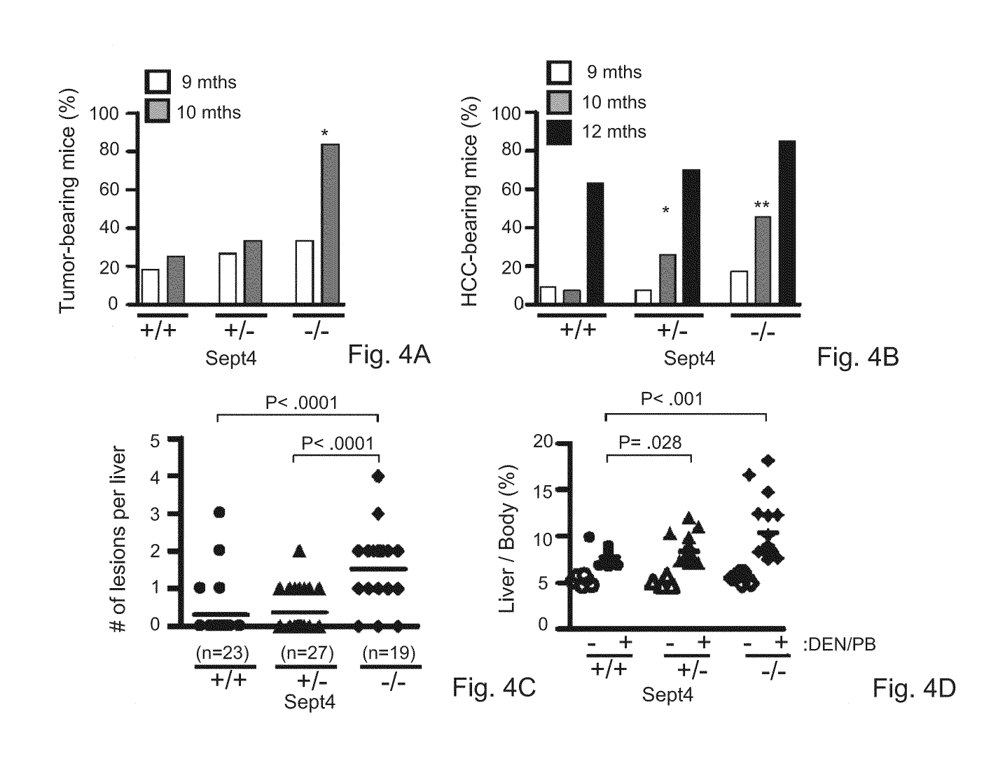

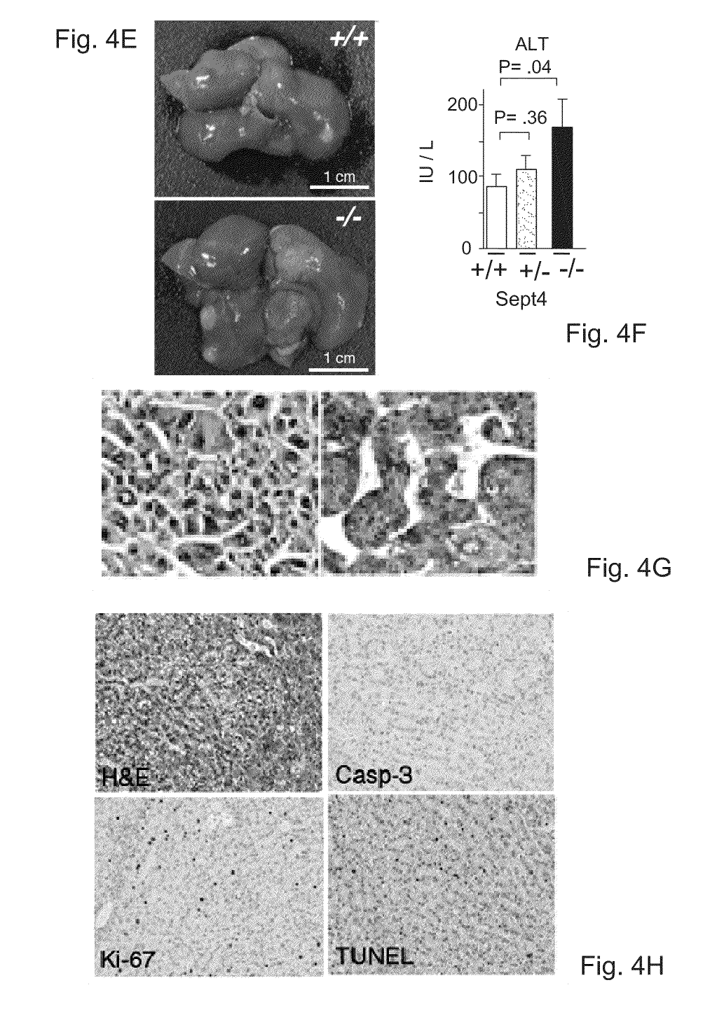

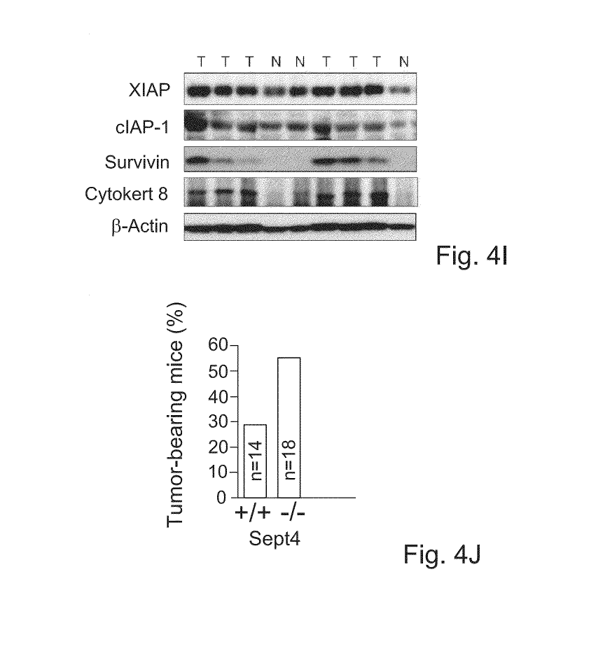

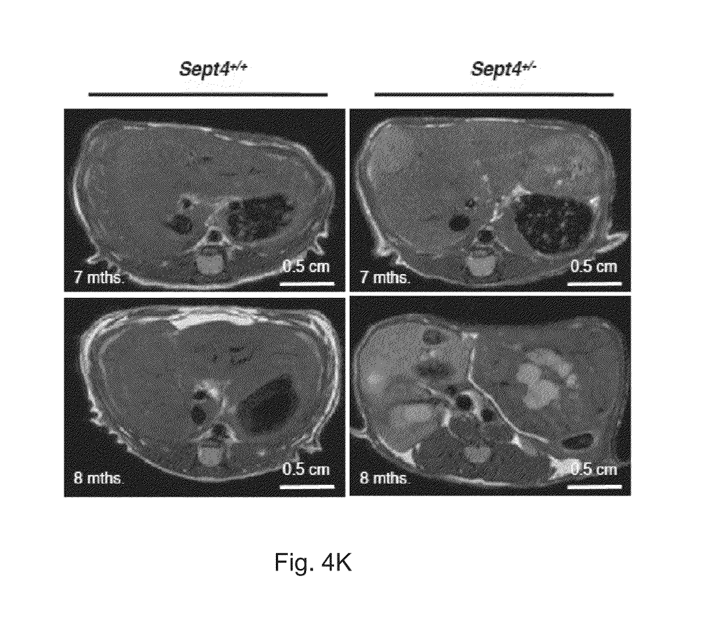

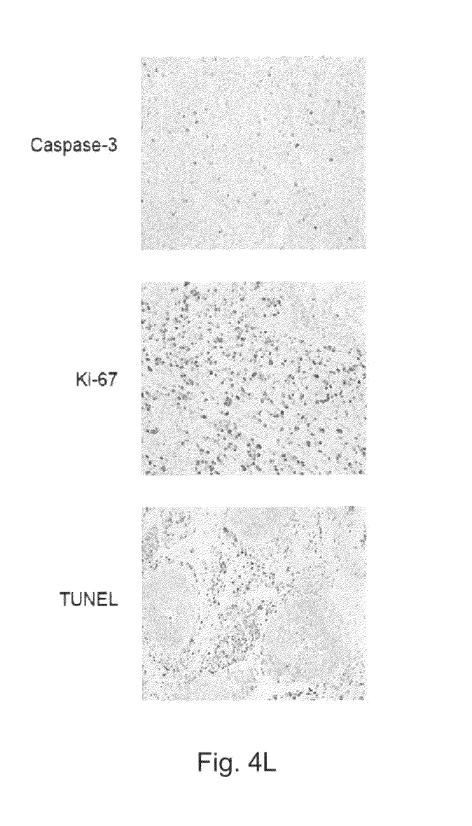

Significantly, the incidence of liver cancer dramatically increased in 9 to 12 month-old Sept4^{-/-} mice when compared to wild-type Sept4^{+/+} mice (FIGS. 4A and 4B). By 10 months of age, more than 80% of the Sept4-deficient mice developed at least one macroscopic lesion (adenoma or carcinoma), compared to only 25% of Sept4^{+/+} mice (FIG. 4A). Upon histological analysis, carcinomas were diagnosed in approximately 50% of Sept4^{-/-} mice but in less than 10% of Sept4^{+/+} mice (FIG. 4B). Moreover, Sept4^{-/-} mice tended to have more and larger tumors, and a higher liver-to-body weight ratio in comparison to their wild-type littermates (FIGS. 4C, D, and E, respectively).

Consistently, higher serum levels of alanine aminotransferase (ALT) reflected greater hepatic injury in Sept4^{-/-} than Sept4^{+/+} mice (FIG. 4F). Given these obvious differences in tumor burden between Sept4^{-/-} and Sept4^{+/+}, the overall survival was not measured. Notably, Sept4 mice showed an overall intermediate vulnerability (FIGS. 4A-4F and 4K), indicating genetic haplo-insufficiency. In Sept4^{-/-} mice, the carcinomas resembled human HCC, with most displaying solid or trabecular patterns with scant stroma and irregular sinusoidal spaces (FIGS. 4G and 4H). Also like human HCC, neoplastic cells were variably sized, had large nuclei with prominent nucleoli (FIG. 4G).

The Sept4^{-/-} HCCs displayed Ki-67 immunohistochemical (IHC) staining, indicative of increased mitotic activity (FIG. 4H), and they were TUNEL-positive yet showed minimal to no caspase-3 activation (FIGS. 4H and 4L), thereby suggesting cell death observed by TUNEL was likely non-apoptotic (34). Accompanying these alterations, the tumors had elevated levels of XIAP, cIAP-1, Survivin, and cytokeratin-8 (FIG. 4I). The inventors therefore concluded that loss of Sept4/ARTS function markedly accelerates hepatocarcinogenesis.

To extend this analysis, the inventors used a genetic mouse model of HCC in which Myc overexpression specifically in the liver under the Albumin promoter causes spontaneous development of HCC (35). Sept4-null mice were bred with transgenic Albumin (Alb)-Myc mice and the rate of tumor development was compared to controls. Again, loss of Sept4 function increased the incidence of HCC development (FIG. 4J). These experiments reveal a tumor suppressor function of Sept4 in two different mouse models of HCC.

### Example 4

### Sept4/ARTS but not Sept4/H5 is the Critical Sept4-Isoform Mediating Tumor Suppressor Function

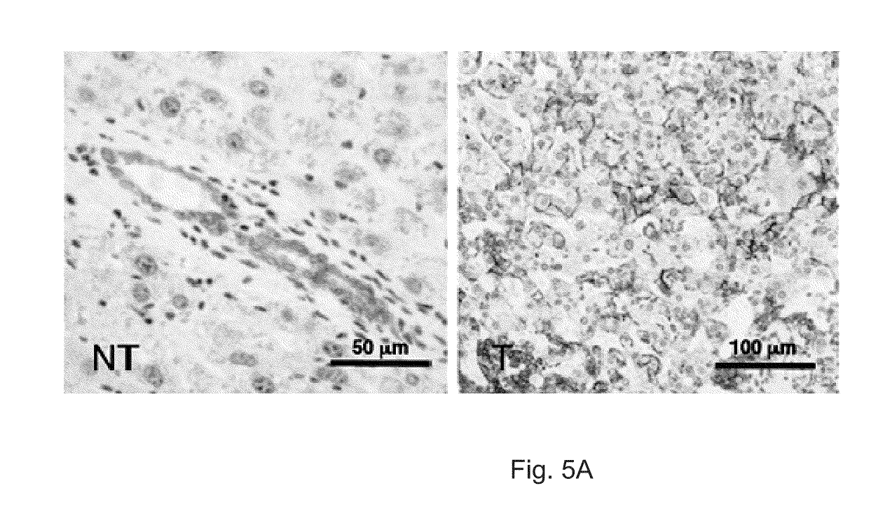

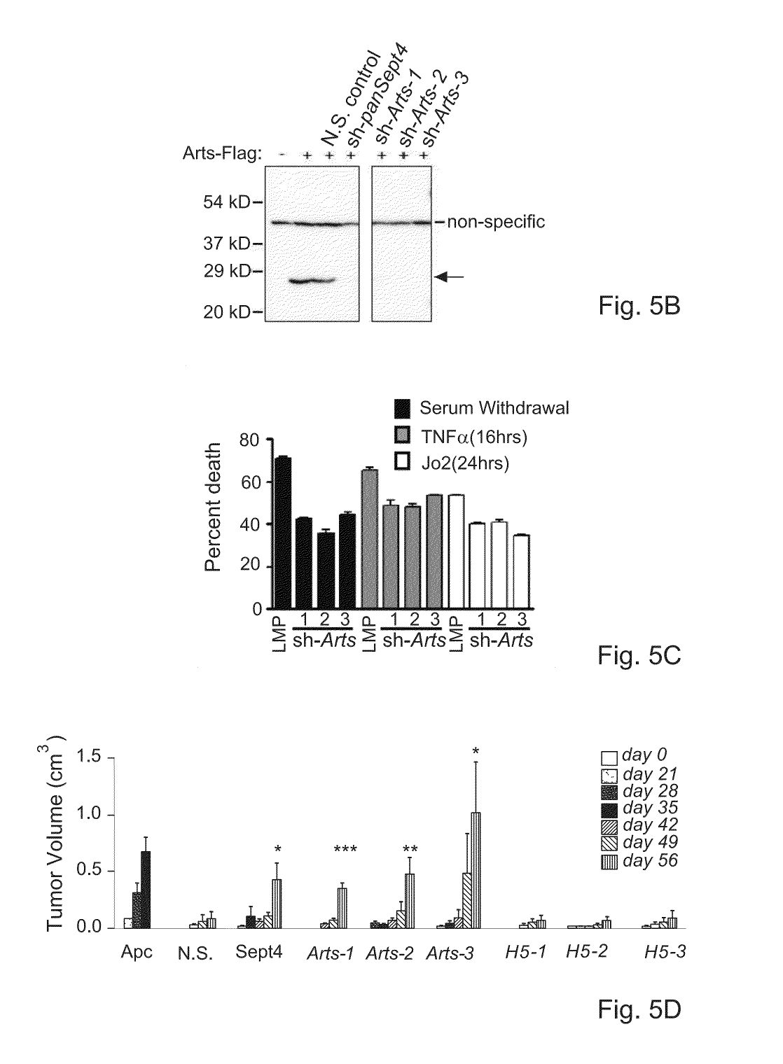

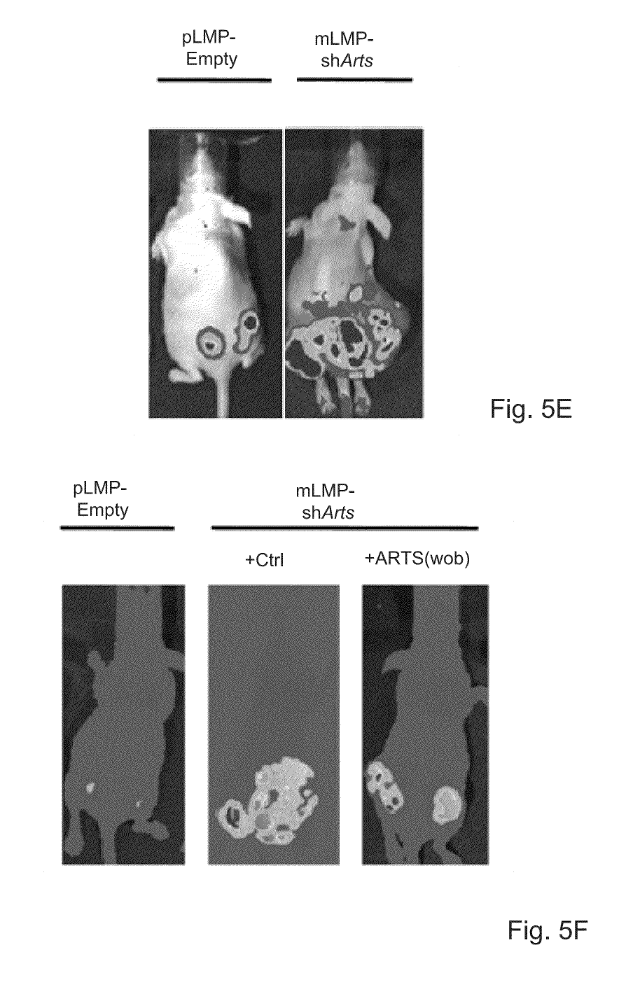

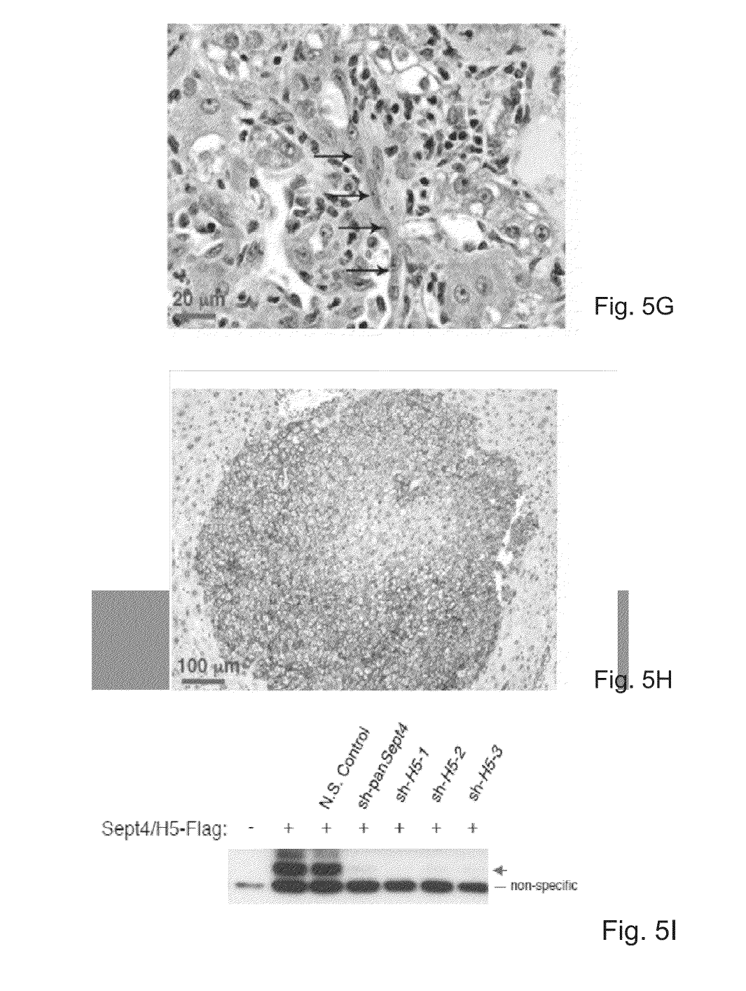

It was further suggested that ARTS confers protective activity cell-autonomously and/or non cell-autonomously. In this regard, often seen adjacent to DEN-PB induced Sept4^{-/-} tumors, proliferation of oval cells, the bipotential stem cell-like cells of the liver is observed (FIGS. 5G and 5H). Upon IHC staining for A6, a marker of oval cells and biliary epithelium, neoplastic cells in many Sept4^{-/-} HCCs were A6-positive (FIGS. 5A, 5G and 5H). This observation raised the possibility that tumor-suppression by Sept4/ARTS involves a regulation liver progenitor cells.

Since Sept4 encodes multiple proteins (FIG. 2A), the contribution of different Sept4-isoforms to tumor suppression was next examined. To this end and taking into consideration the induction of oval cells in the DEN-induced HCCs, liver progenitor cells (hepatoblasts) and short hairpin-RNAs (shRNA)s were used in a transplantation model of oncogenesis (30). ARTS and H5-targeting shRNAs, capable of efficiently knocking-down ARTS or H5, respectively, were identified (FIGS. 5B and 5I). Introduction of ARTS-shRNAs into PHM-1, a hepatoblast cell-line from a p53^{-/-}; Myc background (two genetic lesions frequently observed in HCC), led to an acquisition of cellular resistance against apoptosis induced by serum deprivation, TNFα, or Fas signaling (FIG. 5C). Importantly, the ARTS-shRNAs promoted dramatic tumor growth following sub-cutaneous injection of PHM-1 cells into host mice (FIGS. 5D and 5E). Similarly, a pan-Sept4 hairpin, targeting all Sept4 isoforms, including ARTS (FIGS. 5B and 5I), promoted tumor growth (FIG. 5D). In contrast, Sept4/H5-specific shRNAs failed to promote any growth at all in this system (FIG. 5D).

As control, a non-silencing hairpin and one targeting the genuine tumor suppressor adenomatous polyposis coli (apc), promoted no growth or dramatically accelerated tumorigenesis in this system, respectively (FIG. 5D). Notably, all ARTS-specific hairpins were effective at promoting oncogenesis (FIG. 5D) and the ability of each shRNA to promote tumorigenesis correlated with the ability to knockdown ARTS (FIG. 5B). Furthermore, expression of an ARTS cDNA harboring wobble-mutations (rendering its expression refractory against the shRNA-mediated knockdown) partially rescued tumor growth induced by ARTS-shRNA (FIG. 5F). Based on these results, it was concluded that ARTS is the relevant Sept4-isoform that mediates tumor suppression and that its protective activity is, at least in part, cell-autonomous.

1-34. (canceled)

35. A method for treating, preventing, ameliorating or delaying the onset of a hepatic disorder and associated pathologies or of a solid proliferative disorder in a subject, said method comprises the steps of:
(a) determining in at least one biological sample of said subject at least one of:
(i) the level of expression of Apoptosis Related Protein in the TGF-beta Signaling Pathway (ARTS) and optionally of at least one of Survivin and α-fetoprotein (AFP) to obtain an expression value;
(ii) Sept4/ARTS methylation level of the CpG islands at the TSS to obtain a value of Sept4/ARTS TSS methylation; and
(iii) the level of Histone 3 trimethylation at at least one of lysine 4, lysine 9 and lysine 27 to obtain a trimethylation value of histone H3 at said lysine residues;
(b) determining at least one of:
(i) if the expression value of ARTS obtained in step (a i) is any one of, positive or negative with respect to a predetermined standard expression value of ARTS or the expression value of ARTS in a control sample and optionally, determining if the expression value of at least one of Survivin and AFP is any one of, positive or negative with respect to a predetermined standard expression value of at least one of Survivin and AFP or to the expression value of at least one of Survivin and AFP in a control sample;
(ii) if the value of Sept4/ARTS TSS methylation obtained in step (a ii) is any one of, positive or negative with respect to a predetermined standard Sept4/ARTS TSS methylation or to the Sept4/ARTS TSS methylation value in a control sample;
(iii) if trimethylation value of histone H3 at said lysine residues obtained in step (a iii) is any one of, positive or negative with respect to a predetermined standard trimethylation value of histone H3 or to the trimethylation value in a control sample; and
(c) administering to a subject displaying at least one of (i) a negative expression value of ARTS and optionally, a positive expression value of at least one of Survivin and AFP; (ii) a positive value of Sept4/ARTS TSS methylation; and (iii) a negative trimethylation value of histone H3 at lysine 4 or a positive value at lysine 9 or lysine 27 as determined in step (b), a therapeutically effective amount of at least one chromatin modifying drug or ARTS or any fragment, peptide, analogues and derivatives thereof or any composition comprising the same.

36. The method according to claim 35, wherein the at least one chromatin-modifying drug is a drug that inhibits DNA methylation.

37. The method according to claim 35, wherein said hepatic disorder and associated pathologies is any one of HCC, hepatitis, NASH, fatty liver disease, hepatic steatosis, the metabolic syndrome, steatohepatitis, liver cirrhosis and liver fibrosis, and wherein the solid proliferative disorder is any one of carcinoma, melanoma, sarcoma, glioma and blastoma.

38. The method according to claim 35, wherein the methylation level of the CpG islands at the Sept4/ARTS TSS is determined between positions +249 and +443.

39. The method according to claim 35, wherein methylation is determined in at least one position of +329, +339 and +406 of Sept4/ARTS TSS.

40. The method according to claim 35, wherein determining the level of expression of ARTS and optionally of at least one of Survivin and AFP in a biological sample of said subject is performed by the step of contacting detecting molecules specific for ARTS and optionally for at least one of Survivin and AFP with a biological sample of said subject, or with any nucleic acid or protein product obtained therefrom.

41. The method according to claim 35, wherein determining the level of Sept4/ARTS methylation level of the CpG islands at the TSS in a biological sample of said subject is performed by a methylation specific PCR of bisulfite-treated genomic DNA obtained from said sample.

42. The method according to claim 35, wherein determining the level of Histone 3 trimethylation of at least one of lysine 4, lysine 9 and lysine 27 in a biological sample of said subject is performed by a chromatin immuno-precipitation assay of extracts of said sample.

43. The method according to claim 35, wherein said biological sample is any one of a tissue biopsy and a blood sample.

44. A kit comprising means for performing at least two of:
(a) determining the level of expression of ARTS and optionally of at least one of Survivin and AFP in a biological sample;
(b) determining the level of Sept4/ARTS methylation of the CpG islands at the TSS in a biological sample; and
(c) determining the level of Histone 3 trimethylation of at least one of lysine 4, lysine 9 and lysine 27 in a biological sample.

45. The kit according to claim 44, wherein said means for determining the level of expression of ARTS and optionally of at least one of Survivin and AFP comprise:
(a) detecting molecules specific for ARTS;
(b) optionally, detecting molecules specific for at least one of Survivin and AFP;
(c) predetermined calibration curve providing standard expression values of ARTS;
(d) optionally, predetermined calibration curve providing standard expression values of at least one of Survivin and AFP.

46. The kit according to claim 44, wherein said means for determining the level of Sept4/ARTS methylation of the CpG islands at the TSS comprise:
(a) methylation specific primers;
(b) optionally, reagents for extracting genomic DNA from a sample;
(c) bisulfite containing reagent; and
(d) predetermined calibration curve providing standard values of Sept4/ARTS TSS methylation.

47. The kit according to claim 44, wherein said means for determining the level of Histone 3 trimethylation of at least one of lysine 4, lysine 9 and lysine 27, comprise:
(a) at least one of anti-H3K4me3, anti-H3K27me3 and anti-H3K9me3 antibodies;
(b) primers for amplifying genomic intervals of Sept4/ARTS; and
(c) predetermined calibration curve providing standard values of Histone 3 trimethylation of at least one of lysine 4, lysine 9 and lysine 27.

48. The kit according to claim 44, for use in any one of:
(a) a method for the diagnosis and prognosis of hepatic disorder and associated pathologies or of a solid proliferative disorder in a mammalian subject;
(b) in monitoring and early diagnosis of relapse of a hepatic disorder and associated pathologies or of a solid proliferative disorder in said subject; and
(c) in a method for determining the efficacy and assessing responsivness of a mammalian subject suffering from a hepatic disorder and associated pathologies or of a solid proliferative disorder to treatment with a therapeutic agent.

49. The kit according to claim 44, further comprising at least one of: a therapeutically effective amount of at least one chromatin modifying drug; and ARTS or any fragment, peptide, analogues and derivatives thereof or any composition comprising the same.

50. The kit according to claim 49, for use in a method for treating, preventing, ameliorating or delaying the onset of a hepatic disorder and associated pathologies or of a solid proliferative disorder in a subject in need thereof.

51. The kit according to claim 50, wherein the at least one chromatin-modifying drug is a drug that inhibits DNA methylation.

52. A method for the diagnosis and prognosis of a hepatic disorder and associated pathologies or of a solid proliferative disorder in a mammalian subject, said method comprises the steps of:
(a) determining in at least one biological sample of said subject at least one of:
(i) the level of expression of Apoptosis Related Protein in the TGF-beta Signaling Pathway (ARTS) and optionally of at least one of Survivin and α-fetoprotein (AFP) to obtain an expression value;
(ii) Sept4/ARTS methylation level of the CpG islands at the transcription start site (TSS) to obtain a value of Sept4/ARTS TSS methylation, and
(iii) the level of Histone 3 trimethylation at at least one of lysine 4, lysine 9 and lysine 27 to obtain a trimethylation value of histone H3 at said lysine residues;
(b) determining at least one of:
(i) if the expression value of ARTS obtained in step (a i) is any one of, positive or negative with respect to a predetermined standard expression value of ARTS or to the expression value of ARTS in a control sample and optionally, determining if the expression value of at least one of Survivin and AFP is any one of, positive or negative with respect to a predetermined standard expression value of at least one of Survivin and AFP or to the expression value of at least one of Survivin and AFP in a control sample;
(ii) if the value of Sept4/ARTS TSS methylation obtained in step (a ii) is any one of, positive or negative with respect to a predetermined standard Sept4/ARTS TSS methylation or the Sept4/ARTS TSS methylation value in a control sample; and
(iii) if trimethylation value of histone H3 at said lysine residues obtained in step (a iii) is any one of, positive or negative with respect to a predetermined standard trimethylation value of histone H3 or to the trimethylation value in a control sample;
wherein at least one of: (i) a negative expression value of ARTS and optionally, a positive expression value of at least one of Survivin and AFP; (ii) a positive value of Sept4/ARTS TSS methylation; and (iii) a negative trimethylation value of histone H3 at lysine 4 or a positive value at lysine 9 or lysine 27; indicates that said subject is suffering from a hepatic disorder and associated pathologies or of a solid proliferative disorder.

53. The method according to claim 52, for monitoring and early diagnosis of relapse of said disorder in said subject, the method further comprises the steps of:
(c) repeating step (a) for at least one more temporally-separated test sample of said subject to obtain at least one of (i) the expression value of ARTS and optionally of at least one of Survivin and AFP; (ii) the value of Sept4/ARTS TSS methylation; and (iii) trimethylation value of histone H3 at said lysine residues, for said at least one temporally separated sample;
(d) calculating the rate of change of at least one of (i) the expression value of ARTS and optionally of at least one of Survivin and AFP; (ii) the value of Sept4/ARTS TSS methylation; and (iii) trimethylation value of histone H3 at said lysine residues between said samples;
(e) determining if the rate of change calculated in step (d i-iii) is positive or negative with respect to a standard rate of change determined for a population of subjects suffering from said disorder in relapse and in remission or the rate of change obtained from at least one control sample;
wherein at least one of: (i) a negative rate of change of said expression value of ARTS and optionally, a positive rate of change of said expression value of at least one of Survivin and AFP; (ii) a positive rate of change in the value of Sept4/ARTS TSS methylation; and (iii) a negative rate of change in the trimethylation value of histone H3 at lysine 4 or a positive rate of change at lysine 9 or lysine 27; indicates that said subject is in relapse, thereby monitoring disease progression or providing an early prognosis for disease relapse.

54. The method according to claim 52, for determining the efficacy and assessing responsiveness of a mammalian subject suffering from a hepatic disorder and associated pathologies or a solid proliferative disorder to treatment with a therapeutic agent, said method comprises the step of:
(a) determining in a biological sample of said subject obtained prior to initiation of treatment at least one of:
(i) the level of expression of Apoptosis Related Protein in the TGF-beta Signaling Pathway (ARTS) and optionally of at least one of Survivin and α-fetoprotein (AFP) to obtain an expression value;
(ii) Sept4/ARTS methylation level of the CpG islands at the TSS to obtain a value of Sept4/ARTS TSS methylation; and
(iii) the level of Histone 3 trimethylation at at least one of lysine 4, lysine 9 and lysine 27 to obtain a trimethylation value of histone H3 at said lysine residues;
(b) repeating step (a) in at least one other biological sample of said subject obtained after initiation of said treatment,
(c) calculating the rate of change of at least one of (i) the expression value of ARTS and optionally of at least one of Survivin and AFP; (ii) the value of Sept4/ARTS TSS methylation; and (iii) trimethylation value of histone H3 at said lysine residues between samples obtained before and after initiation of said treatment; and
(d) determining if the rate of change calculated in step (c i-iii) is positive or negative with respect to a standard rate of change determined for a population of responder or and non-responder subjects suffering from said disorder and treated with said therapeutic agent or the rate of change obtained from at least one control sample;
wherein at least one of: (i) a negative rate of change of said expression value of ARTS and optionally, a positive rate of change of said expression value of at least one of Survivin and AFP; (ii) a positive rate of change in the value of Sept4/ARTS TSS methylation; and (iii) a negative rate of change in the trimethylation value of histone H3 at lysine 4 or a positive value at lysine 9 or lysine 27; indicates that said subject belongs to a pre-established population associated with lack of responsiveness to treatment with said therapeutic agent.
